(19) United States
(12) Patent Application Publication
(10) Pub. No.: US 2025/0134903 A1
(43) Pub. Date: May 1, 2025

(54) COMPOSITIONS AND METHODS FOR TREATING PAIN

(71) Applicant: UNDAUNTED BIO, INC., Pittsburgh, PA (US)

(72) Inventors: ERNEST K. MANDERS, Pittsburgh, PA (US); Mark Anthony David COLLINS, Pittsburgh, PA (US)

(73) Assignee: UNDAUNTED BIO, INC., Pittsburgh, PA (US)

(21) Appl. No.: 18/934,671

(22) Filed: Nov. 1, 2024

Related U.S. Application Data
(60) Provisional application No. 63/595,300, filed on Nov. 1, 2023.

(51) Int. Cl. A61K 31/5415; A61K 9/00; A61K 9/20; A61P 25/02

(52) U.S. Cl. CPC A61K 31/5415; A61K 9/006; A61K 9/20; A61P 25/02

(57) ABSTRACT

The present disclosure describes methods of treating conditions such as trigeminal neuralgia and pain related thereto. The methods of the present disclosure may include administering to a patient in need thereof a therapeutically effective amount of Trimeprazine or a pharmaceutically acceptable salt thereof, thereby treating the patient. The methods may include reducing a patient's pain as determined by patient-reported or clinician-delivered outcome assessments.

### CROSS-REFERENCE TO RELATED APPLICATIONS

This application claims priority to U.S. Provisional Patent Application No. 63/595,300, filed on Nov. 1, 2023, which is hereby incorporated by reference herein in its entirety.

### BACKGROUND

Trigeminal neuralgia (TN), also called tic douloureux, is a chronic pain condition that affects the trigeminal or fifth cranial nerve, one of the most widely distributed nerves in the head. TN is a form of neuropathic pain (pain associated with nerve injury or nerve lesion.) The typical or “classic” form of the disorder (called “Type 1” or TN1) causes extreme, sporadic, sudden burning or shock-like facial pain that lasts anywhere from a few seconds to as long as two minutes per episode. These attacks can occur in quick succession, in volleys lasting as long as two hours. The “atypical” form of the disorder (called “Type 2” or TN2), is characterized by constant aching, burning, stabbing pain of somewhat lower intensity than Type 1. Both forms of pain may occur in the same person, sometimes at the same time. The intensity of pain can be physically and mentally incapacitating.

The trigeminal nerve is the fifth cranial nerve (Cranial Nerve V) and is one of 12 pairs of nerves that are attached to the brain. The nerve has three branches emanating from the “Gasserian Ganglion” (semilunar ganglion) that conduct sensations from the upper, middle, and lower portions of the face, as well as the oral cavity, to the brain. The ophthalmic, or upper, branch (V1) supplies sensation to most of the scalp, forehead, and front of the head. The maxillary, or middle, branch (V2) stimulates the cheek, upper jaw, top lip, teeth and gums, and to the side of the nose. The mandibular, or lower, branch (V3) supplies nerves to the lower jaw, teeth and gums, and bottom lip. More than one nerve branch can be affected by the disorder. Rarely, both sides of the face may be affected at different times in an individual, or even more rarely at the same time (called bilateral TN).

Pain varies, depending on the type of TN, and may range from sudden, severe, and stabbing to a more constant, aching, burning sensation. The intense flashes of pain (paraxosyms) can be triggered by seemingly innocuous stimuli that cause vibration or contact with the cheek (such as when shaving, washing the face, or applying makeup), brushing teeth, eating, drinking, talking, or being exposed to the wind. The pain may affect a small area of the face or may spread. Bouts of pain rarely occurs at night when the affected individual is sleeping. TN is typified by attacks that stop for a period of time and then return, but the condition can be progressive, with attacks often worsening over time, with fewer and shorter pain-free periods before they recur. Eventually, the pain-free intervals disappear and medication to control the pain becomes less effective highlighting the need for more effective treatment regimens for managing TN.

### SUMMARY

In some aspects, there is embodied a method for the treatment of the pain associated with trigeminal neuralgia comprising administering to a patient in need thereof a therapeutically effective amount of Trimeprazine or a pharmaceutically acceptable salt thereof, thereby treating the trigeminal neuralgia in the patient.

In some aspects, the therapeutically effective amount of Trimeprazine or a pharmaceutically acceptable salt thereof is administered once a day.

In some aspects, the therapeutically effective amount of Trimeprazine or a pharmaceutically acceptable salt thereof is administered twice a day.

In some aspects, the therapeutically effective amount of Trimeprazine or a pharmaceutically acceptable salt thereof is administered via oral administration, intravenous administration (IV), intramuscular administration (IM), intrathecal administration, subcutaneous administration (SC), sublingual or buccal administration, rectal transdermal administration, vaginal administration, cutaneous administration, administration, nasal administration, or combinations thereof.

In some aspects, the therapeutically effective amount of Trimeprazine or a pharmaceutically acceptable salt thereof is administered via oral administration.

In some aspects, the therapeutically effective amount of Trimeprazine or a pharmaceutically acceptable salt thereof is administered via an oral thin film formulation.

In some aspects, the therapeutically effective amount of Trimeprazine or a pharmaceutically acceptable salt thereof is about 0.5 mg to about 80 mg.

In some aspects, the therapeutically effective amount of Trimeprazine or a pharmaceutically acceptable salt thereof is about 2.5 to about 5 mg.

In some aspects, the therapeutically effective amount of Trimeprazine or a pharmaceutically acceptable salt thereof is about 0.5 to about 2.5 mg.

In some aspects, the treatment of the pain associated with trigeminal neuralgia comprises a reduction in the frequency of pain, a reduction in the duration of pain, a reduction in the intensity of pain, or any combination thereof.

In some aspects, a reduction in the duration of pain comprises a reduction in the duration of a single episode of pain, a reduction in the duration of a series of episodes of pain, and any combination thereof.

In some aspects, a series of episodes of pain comprises at least two separate episodes of pain.

In some aspects, the pain associated with trigeminal neuralgia is stabbing pain, lancinating pain, shock-like pain, electric shock-like pain, constant aching, burning, stabbing pain of somewhat lower intensity than Type 1 TN, and any combination thereof.

In some aspects, a reduction in the frequency of pain, a reduction in the duration of pain, a reduction in the intensity of pain, or any combination thereof is measured using a patient diary, a PGIC score, a MSQ score, a BNI Pain Intensity score, a Penn-FPS-R score, a Penn-FPS score, a EQ-5D-5L score, a WPAI score or any combination thereof.

In some aspects, administering the therapeutically effective amount of Trimeprazine or a pharmaceutically acceptable salt thereof comprises administering an initial dose of between 2.5 to about 5.0 mg.

In some aspects, administering the therapeutically effective amount of Trimeprazine or a pharmaceutically acceptable salt thereof comprises administering an initial dose of between 0.5 to about 2.5 mg.

Some aspects further comprise administering an additional therapeutically effective amount of Trimeprazine or a pharmaceutically acceptable salt thereof, about every 30 minutes to about every 5 hours following the initial dose if treatment of pain associated with trigeminal neuralgia is not achieved.

In some aspects, the additional therapeutically effective amount of Trimeprazine or a pharmaceutically acceptable salt thereof is increased about every 30 minutes to about every 5 hours until treatment of the pain associated with trigeminal neuralgia is achieved.

In some aspects, once treatment of pain associated with trigeminal neuralgia is achieved, the additional therapeutically effective amount of Trimeprazine or a pharmaceutically acceptable salt thereof is maintained as the minimum effective level used for the treatment of pain associated with trigeminal neuralgia.

In some aspects, the minimum effective level of therapeutically effective amount of Trimeprazine or a pharmaceutically acceptable salt thereof is administered until treatment of pain treatment of pain associated with trigeminal neuralgia is no longer achieved, after which the minimum effective level of therapeutically effective amount of Trimeprazine or a pharmaceutically acceptable salt thereof may be further increased until treatment of pain treatment of pain associated with trigeminal neuralgia is once again achieved.

In some aspects, at least about every 3 months, the therapeutically effective amount of Trimeprazine or a pharmaceutically acceptable salt thereof administered is decreased to the minimum effective level of therapeutically effective amount of Trimeprazine or a pharmaceutically acceptable salt thereof.

In some aspects, at least about every 3 months, administration of the therapeutically effective amount of Trimeprazine or a pharmaceutically acceptable salt thereof administered is discontinued.

### BRIEF DESCRIPTION OF DRAWINGS

Aspects, features, benefits, and advantages of the embodiments described herein will be apparent with regard to the following description, appended claims, and accompanying drawings where:

### DETAILED DESCRIPTION

Various aspects now will be described more fully hereinafter. Such aspects may, however, be exemplified in many different forms and should not be construed as limited to the examples set forth herein; rather, these examples are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 mg to 8 mg is stated, 2 mg, 3 mg, 4 mg, 5 mg, 6 mg, and 7 mg are also intended to be explicitly disclosed, as well as the range of values greater than or equal to 1 mg and the range of values less than or equal to 8 mg.

All percentages, parts and ratios are based upon the total weight of the topical compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value and also includes the exact value disclosed, e.g., “about 50” means 45 to 55, “about 25,000” means 22,500 to 27,500, etc., unless the context of the disclosure indicates otherwise, or is inconsistent with such an interpretation. For example, in a list of numerical values such as “about 49, about 50, about 55,” “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subject.

The term “carrier” as used herein encompasses carriers, excipients, and diluents, meaning a material, composition, or vehicle, such as a liquid or solid filler, diluent, excipient, solvent, or encapsulating material involved in carrying or transporting a pharmaceutical, cosmetic or other agent across a tissue layer such as the stratum corneum or stratum spinosum.

The term “disorder” as used herein means, and is used interchangeably with, the terms disease, condition, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject and/or enhance the texture, appearance, color, sensation, and/or hydration of the intended tissue treatment area. The actual amount which comprises the “effective amount” or “therapeutically effective amount” will vary depending on a number of conditions including, but not limited to, the severity of the disorder, the size and health of the patient, the route of administration, and combinations thereof. A skilled medical practitioner can readily determine the “effective amount” or the “therapeutic amount” using methods known in the medical arts.

The term “modified release” as used herein refers to pharmaceutical compositions that do not otherwise release the entirety of the active ingredient immediately. For example, it may release the active ingredient at a sustained or controlled rate over an extended period of time or may release the active ingredient after a lag time after administration, or may be used optionally in combination with an immediate release composition. Modified release includes extended release, sustained release, controlled release, and delayed release. The term “extended release” or “sustained release” as used herein is a dosage form that makes a drug available over an extended period of time after administration relative to a dose delivered in an entirely immediate release form. The term “delayed release” as used herein is a dosage form that releases a drug at a time other than immediately upon administration. The terms “orally” and “oral” and “oral administration” as used herein refer to the route of administration where a substance is taken through the mouth.

The phrase “pharmaceutically acceptable” is used herein to refer to those agents of interest/compounds, salts, compositions, dosage forms, etc., which are within the scope of sound medical judgment, suitable for use in contact with the tissues of human beings and/or other mammals without excessive toxicity, irritation, allergic response, and/or other problem or complication, commensurate with a reasonable benefit/risk ratio. In some aspects, “pharmaceutically acceptable” means approved by a regulatory agency of the federal or a state government or listed in the U.S. Pharmacopeia or other generally recognized pharmacopeia for use in mammals (e.g. animals), and more particularly, in humans.

The term “salts” as used herein embraces pharmaceutically acceptable salts commonly used to form alkali metal salts of free acids and to form additional salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Non-limiting examples of suitable inorganic acids include hydrochloric, hydrobromic, hydroiodic, nitric, carbonic, sulfuric, and phosphoric acid. Appropriate organic acids can be selected from: aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylate, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, alginic, 3-hydroxybutyric, galactaric, and galacturonic acid.

The term “patient” and “subject” are interchangeable as used herein and may be taken to mean any living organism which may be treated with compounds of the present disclosure. As such, the terms “patient” and “subject” may include, but are not limited to, any non-human mammal, primate or human. In some aspects, the “patient” or “subject” is a mammal; exemplary mammals include: mice, rats, rodents besides mice and rats, rabbits, dogs, cats, swine, cattle, sheep, horses, primates, or humans. In some aspects, the patient or subject is an adult, child or infant. In some aspects, the patient or subject is a human.

The term “treating” is used herein, for instance, in reference to methods of treating a disorder or a systemic condition, and generally include the administration of a compound or composition which reduces the frequency of, or delays the onset of, symptoms of a medical condition and/or enhances the texture, appearance, color, sensation, and/or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition. This can also include administration of a compound or composition which results in a reduction in the frequency of pain, a reduction in the duration of pain, a reduction in the intensity of pain, or any combination thereof. In some embodiments, a reduction in the duration of pain comprises a reduction in the duration of a single episode of pain, a reduction in the duration of a series of episodes of pain, and any combination thereof. In some aspects, a series of episodes of pain comprises at least two separate episodes of pain. In some aspects, the pain associated with trigeminal neuralgia is stabbing pain, lancinating pain, shock-like pain, electric shock-like pain, constant aching, burning, stabbing pain of somewhat lower intensity than Type 1 TN, and any combination thereof. In some aspects, a reduction in the frequency of pain, a reduction in the duration of pain, a reduction in the intensity of pain, or any combination thereof is measured using a patient diary, a PGIC score, a MSQ score, a BNI Pain Intensity score, a Penn-FPS-R score, a Penn-FPS score, a EQ-5D-5L score, a WPAI score or any combination thereof.

As used herein, the term “pharmaceutically acceptable carrier” refers to any of the standard pharmaceutical carriers including, but not limited to non-toxic solvent, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintrigrants (e.g., potato starch or sodium starch glycolate), and the like. The compositions also can include stabilizers and/or preservatives.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Various aspects of the disclosure are directed to compositions containing a phenothiazine for treating pain and methods for using such compositions to treat, prevent, and/or ameliorate pain. Such compositions may further include various excipients that facilitate oral, topical, or both oral and topical administration. The compositions and methods described herein may reduce pain and other symptoms associated with chronic conditions.

The phenothiazine may include, for example, chlorpromazine, diethazine, ethopropazine, fluphenazine, methdilazine, perphenazine, prochlorperazine, promazine, promethazine, mesoridazine, thiethylperazine, thioridazine, trifluoperazine, triflupromazine, Trimeprazine, and the like, various derivatives and salts thereof, and combinations thereof. In certain aspects, the phenothiazine may be one or more of diethazine, ethopropazine, methdilazine, promethazine, thiethylperazine, or Trimeprazine, and in some aspects, the phenothiazine may be Trimeprazine or a pharmaceutically acceptable salt thereof.

The person of ordinary skill in the art will understand and appreciate the dosages and timing of said dosages to be administered to a patient in need thereof. The doses and duration of treatment may vary and may be based on assessment by one of ordinary skill in the art based on monitoring and measuring improvement in the underlying malady. This assessment may be made based on outward physical signs of improvement, such as reduction in pain. The doses may also depend on the condition or disease being treated, the degree of the condition or disease being treated and further on the age and weight of the patient. In some aspects, the amount of phenothiazine administered using the exemplified methods aspects can vary and may be, for example, from about 0.05 mg/kg/day to about 5 mg/kg/day. In some aspects, the amount of phenothiazine delivered may be from about 0.05 mg/kg/day to about 5 mg/kg/day, from about 0.1 mg/kg/day to about 5 mg/kg/day, from about 0.5 mg/kg/day to about 5 mg/kg/day, from about 1 mg/kg/day to about 5 mg/kg/day, from about 1.5 mg/kg/day to about 5 mg/kg/day, from about 0.1 mg/kg/day to about 3 mg/kg/day, or any range or individual value encompassed by these exemplary ranges. The compositions administered may generally include from about 0.5 mg to about 80 mg of phenothiazine, from about 0.5 mg to about 50 mg, from about 1 mg to about 20 mg, from about 1 mg to about 10 mg, from about 1 mg to about 5 mg, or any range or individual dosage encompassed by these exemplary ranges. In some aspects, the compositions may be administered 1, 2, 3, 4, or more times per day resulting in a total daily administration of from about 0.5 mg to about 80 mg per day, from about 0.5 mg to about 40 mg per day, from about 1 mg to about 30 mg per day, from about 1 mg to about 20 mg per day or any individual amount or range encompassed by these exemplary ranges.

Specific modes of administration will depend on the indication. The selection of the specific route of administration and the dose regimen may be adjusted or titrated by the clinician according to methods known to the clinician to obtain the optimal clinical response. The amount of compound to be administered may be that amount which is therapeutically effective. The dosage to be administered may depend on the characteristics of the subject being treated, for example, the particular animal or human subject treated, age, weight, health, types of concurrent treatment, if any, and frequency of treatments, and can be easily determined by one of skill in the art (e.g., by the clinician).

The exemplary pharmaceutical compositions described herein may be prepared for administration by a variety of different routes. In general, the type of carrier is selected based on the mode of administration. The compositions of various aspects can be formulated for systemic delivery or local delivery to affected tissue. For example, in some aspects, the compositions may be formulated for systemic delivery by oral administration, intravenous administration (IV), intramuscular administration (IM), intrathecal administration, subcutaneous administration (SC), sublingual or buccal administration, rectal administration, vaginal administration, cutaneous administration, transdermal administration, nasal administration, and the like and combinations thereof. In other aspects, the compositions described herein can be delivered locally using, for example, topical administration.

For oral administration, the compounds can be formulated readily by combining these compounds with pharmaceutically acceptable carriers well known in the art. As used herein, the term “pharmaceutically acceptable carrier” means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers include: sugars, such as lactose, glucose and sucrose; starches such as corn starch and potato starch; cellulose and its derivatives, such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations, and combinations thereof. Such carriers may enable the compounds described herein to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropyl-methylcellulose, sodium carboxymethylcellulose, and polyvinylpyrrolidone (PVP) and combinations thereof. If desired, disintegrating agents can be added, such as, but not limited to, the cross-linked polyvinyl pyrrolidone, agar, or alginic acid or a salt thereof such as sodium alginate, and combinations thereof.

Dragee cores can be provided with suitable coatings. For this purpose, concentrated sugar solutions can be used, which can optionally contain gum arabic, talc, polyvinyl pyrrolidone, carbopol gel, polyethylene glycol, titanium dioxide, lacquer solutions, and suitable organic solvents or solvent mixtures, and combinations thereof. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols, and combinations thereof. In addition, stabilizers can be added. All formulations for oral administration should be in dosages suitable for such administration.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Syrups and elixirs may be formulated with sweetening agents, including for example glycerol, propylene glycol, sorbitol, sucrose and combinations thereof. Such formulations may also contain a demulcent, a preservative, a flavoring agent, a coloring agent and combinations thereof.

For buccal or sublingual administration, the compositions can take the form of tablets, flash melts or lozenges formulated in any conventional manner.

In addition to the formulations described previously, the compounds described herein can also be formulated as a depot preparation. Such long-acting formulations can be administered by implantation (for example subcutaneously or intramuscularly) or by intramuscular injection.

Depot injections can be administered at intervals of from about 1 to about 6 months or longer intervals. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

In transdermal administration, the compounds described herein, for example, can be applied to a plaster, or can be applied by transdermal, therapeutic systems that are consequently supplied to the organism.

In some aspects, an exemplary pharmaceutical composition may be associated with fibers. For example, in some aspects, a fiber-containing composition may include electrospun polymers having the phenothiazine associated with the electrospun polymers. In some aspects, the phenothiazine may be dispersed within the electrospun polymer and may exclude pharmaceuticals on the outer surface of the fibers formed from the electrospun polymer. Pharmaceuticals dispersed within the electrospun polymer may provide the added benefit of being resistant to accidental and/or unanticipated removal of the phenothiazine from the fiber. In other aspects, the phenothiazine may be additionally or alternatively associated with an outer surface of the fiber by, for example, dipping, spraying, or otherwise treating the outside surface of a fiber with the pharmaceutical.

The electrospun polymers of some aspects may include one or more polymers. In some aspects, the polymer may be a water-soluble polymer or a combination of water-soluble polymers. In particular aspects, the one or more polymers may include a combination of synthetic polymers and naturally occurring polymers in any combination and/or compositional ratio.

In some aspects, the phenothiazine associated with the polymer may be dispersed or dissolved in an oil such as, for example and oil selected from: cannabis oil, cannabidiol (CBD) oil, olive oil, sesame oil, canola oil, palm oil, vegetable oil, derivatives thereof, or combinations thereof. In some aspects, the pharmaceutical may have a crystalline form. In further aspects, the phenothiazine may be a crystal dispersed or dissolved in an oil or a solution.

Pharmaceutical and therapeutic compositions of the compounds can also include suitable solid or gel phase carriers or excipients. Examples of such carriers or excipients include, but are not limited to, calcium carbonate, calcium phosphate, various sugars, starches, cellulose derivatives, gelatin, polymers such as, e.g., polyethylene glycols, and combinations thereof.

In various aspects, the compositions may further include pharmaceutical and/or cosmetically acceptable carries, excipients, diluents, fillers, disintegrants, desiccants, binders, lubricants, surfactants, hydrophobic vehicles, water soluble vehicles, emulsifiers, buffers, humectants, moisturizers, solubilizers, preservatives, colorants, plasticizers, carriers, or combinations thereof. The person of ordinary skill in the art can refer to various pharmacologic references such as, for example, Modern Pharmaceutics, Banker & Rhodes, Marcel Dekker, Inc. (1979) and Goodman & Gilman's The Pharmaceutical Basis of Therapeutics, 6th Edition, MacMillan Publishing Co, New York (1980) for guidance in determining the amount of such components in the compositions and formulations of aspects. Any previously mentioned, carries, excipients, diluents, fillers, disintegrants, desiccants, binders, lubricants, surfactants, hydrophobic vehicles, water soluble vehicles, emulsifiers, buffers, humectants, moisturizers, solubilizers, preservatives, colorants, plasticizers, carriers, and combinations thereof may be incorporated into such compositions.

In some aspects, an exemplary topical composition may include a solvent such as: water, isopropyl alcohol, dipropylene glycol methyl-ether, butylated hydroxytoluene dipropylene glycol monomethyl-ether, 1-methoxy 2-propanol (glysolv PM/lcinol PM), ethylene glycol monobutylether, butyl diglysolv 1, transcutol, propylene glycol (PG), N-methyl-2 pyrrolidone (NMP), methylene chloride, diethyl ether, ethanol, acetonitrile, ethyl acetate, benzyl alcohol, a combination of natural oils, ethylene glycol, propylene glycol, dimethyl polysiloxane (DMPX), oleic acid, caprylic acid, 1-octanol, ethanol (denatured or anhydrous), liposomal compositions, suitable plant oils, such as aloe vera derivatives or sesame seed oil and/or derivatives thereof, ethosomes, azone, castor oil derivatives, such as ethoxylated castor oil, jojoba oil derivatives, corn oil derivatives, emu oil derivatives, and the like and combinations thereof. The solvent can be present in any suitable concentration. For example, in some aspects, the solvent may be present at from about 5 wt. % to about 99.9 wt. %, from about 10 wt. % to about 95 wt. %, from about 25 wt. % to about 90 wt. %, from about 20 wt. % to about 80 wt. % of the total composition, or any range or individual concentration of solvent encompassed by these example ranges.

In some aspects, the topical compositions may include a polar water-miscible solvent, such as an alcohol and/or glycol. Polar water-miscible solvents may improve skin penetration and solvation of the active agent. The polar water-miscible solvent may include, for example, C1-C4 alcohols, polyethylene glycol, ethylene glycol, propylene glycol, butylene glycol, pentylene glycol, glycerol, diethylene glycol monoethyl ether, propylene carbonate, and the like and combinations and mixtures thereof. The total amount of polar water-miscible solvent may be less than about 10 wt. % by weight of the total composition or from about 0.5 wt. % to about 10 wt. %, from about 1 wt. % to about 5 wt. %, from about 0.5 wt. % to about 5 wt. %, or any range or individual concentration of solvent encompassed by these exemplary ranges.

In some aspects, the compositions may include a surfactant. The surfactant may be incorporated into the oil phases, the aqueous phase, or both. Suitable surfactants include, for example, alkyl polyglycol ethers, alkyl polyglycol esters, ethoxylated alcohols, polyoxyethylene sorbitan fatty acid esters, polyoxyethylene fatty acid esters, ionic or non-ionic surfactants, hydrogenated castor oil/polyoxyethylene glycol adducts, castor oil/polyoxyethylene glycol adducts, sorbitan fatty acid esters (such as Span 20 or Span 80), block copolymers of ethylene oxides and propylene oxides (such as Pluronic L121 or Pluronic F68), polymeric surfactants having crosslinked copolymers of acrylic acid, such as Pemulen Tr-1 and Pemulen Tr-2, and the like and combinations and mixtures thereof. The composition may include surfactant in a concentration of from about 0.1 wt. % to about 5 wt. %, from about 0.5 wt. % to about 3 wt. %, from about 0.7 wt. % to about 2 wt. %, or any range or individual concentration of solvent encompassed by these exemplary ranges.

In some aspects, the compositions may include an antioxidant. Such antioxidant may be, for example, butylated hydroxytoluene, ascorbic acid, ascorbic palmitate, butylated hydroxyanisole, 2,4,5-tri hydroxybutyrophenone, 4-hydroxymethyl-2,6-di-tert-butyl phenol, erythorbic acid, gum guaiac, propyl gallate, thiodipropionic acid, dilauryl thiodipropionate, tert-butylhydroquinone, tocopherol, and the like and pharmaceutically acceptable salt or ester thereof, or combinations thereof. The antioxidant can be present in a concentration of from about 0.01 wt. % to about 1 wt. % of the total composition or any individual concentration encompassed by this example range.

In some aspects, the composition may include an emulsifying agent including, for example, various monoglycerides, diglycerides, triglycerides, and blends thereof at a concentration of from about 3 wt. % to about 10 wt. % of the total composition.

In some aspects, the composition may further include an analgesic agent such as, for example, methyl salicylate, codeine, morphine, methadone, pethidine, buprenorphine, hydromorphone, levorphanol, oxycodone, fentanyl, a non-steroidal anti-inflammatory drug (NSAID), and the like and combinations thereof. The amount of the analgesic agent in such compositions may be from about 0.01 wt. % to about 5 wt. % of the total composition.

In some aspects, the composition may further include a moisturizing agent. Examples of moisturizing agents of use in exemplary compositions include propylene glycol, glycerin and the like and combinations thereof. The amount of moisturizing agent in such compositions may be from about 0.01 wt. % to about 10 wt. % of the total composition.

In some aspects, the composition may further include a pharmaceutically acceptable buffer sufficient to adjust and maintain the pH of the compositions described herein in the range of from about 7.0 to about 14.0, or from about 8.5 to about 12.0. Typically, suitable buffers include citrate, phosphate, glycine, and the like and combinations thereof. The amount of buffer in such compositions may be from about 0.01 wt. % to about 10 wt. % of the total composition.

In some aspects, exemplary compositions may further contain a mineral, mineral salt, or combinations thereof. Exemplary minerals of use include, but are not limited to, selenium, sulfur, zinc, iron, chlorine, cobalt, copper, manganese, molybdenum, and iodine. The amount of the mineral or mineral salts in exemplary topical formulations includes any therapeutically effective amount. For example, the mineral or mineral salt may have a concentration of from about 0.01 wt. % to about 5 wt. %, relative to the total amount of the composition, from about 0.1 wt. % to about 1 wt. %, relative to the total amount of the composition, or any range or individual concentration encompassed by these example ranges.

In some aspects, the compositions may further include a vitamin or a combination of vitamins. Vitamins are organic molecules that are essential nutrients that organisms need to sustain proper biological function and metabolism. Exemplary of use include, but are not limited to: vitamin A, vitamin B1, vitamin B2, vitamin B3, vitamin B4, vitamin B5, vitamin B6, vitamin B7, vitamin B8, vitamin B9, vitamin Bio, vitamin B11, vitamin B12, vitamin C, vitamin D, vitamin E, and vitamin K. The amount of the vitamin in the topical formulation can be any therapeutically effective amount. For example, the vitamin may have a concentration of from about 0.01 wt. % to about 5 wt. %, relative to the total amount of the composition, from about 0.1 wt. % to about 1 wt. %, relative to the total amount of the composition, or any range or individual concentration encompassed by these example ranges.

In some aspects, exemplary compositions may further include: an anti-inflammatory compound such as hyaluronic acid, curcumin, glutathione, methotrexate, tofacitinib, 6-mercaptopurine, azathioprine sulfasalazine, mesalazine, olsalazine chloroquine/hydroxychloroquine, penicillamine, aurothiomalate (intramuscular and oral), azathioprine, colchicine, corticosteroids (oral, inhaled, and local injection), a beta-2 adrenoreceptor agonist (salbutamol, terbutaline, salmeterol), a xanthine (theophylline, aminophylline), cromoglycate, nedocromil, ketotifen, ipratropium and oxitropium, cyclosporin, FK506, rapamycin, mycophenolate mofetil, leflunomide, an NSAID (e.g. ibuprofen), a corticosteroid (e.g. prednisolone), a phosphodiesterase inhibitor, an adenosine agonist, an antithrombotic agent, a complement inhibitor, an adrenergic agent, an agent that interferes with signaling by proinflammatory cytokines such as TNF or IL-1 (e.g., a NIK, IKK, p38 or MAP kinase inhibitor), an IL-1 converting enzyme inhibitor, a T-cell signaling inhibitor (e.g. a kinase inhibitor), a metalloproteinase inhibitor, sulfasalazine, a 6-mercaptopurine, an angiotensin converting enzyme inhibitor, a soluble cytokine receptor (e.g. soluble p55 or p75 TNF receptors and the derivatives p75TNFRigG (etanercept) and p55TNFRigG (Lenercept), siL-1RI, siL-1RII, siL-6R), an anti-inflammatory cytokine (e.g. IL-4, IL-1 0, IL-11, IL-13 and TGF), celecoxib, folic acid, hydroxychloroquine sulfate, rofecoxib, etanercept, infliximab, adalimumab, certolizumab, tocilizumab, abatacept, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, tramadol HCL, salsalate, sulindac, cyanocobalamin/fa/pyridoxine, acetaminophen, alendronate sodium, prednisolone, cortisone, betamethasone, morphine sulfate, lidocaine hydrochloride, indomethacin, glucosamine sulf/chondroitin, amitriptyline HCl, sulfadiazine, oxycodone HCV acetaminophen, olopatadine HCl misoprostol, naproxen sodium, omeprazole, cyclophosphamide, rituximab, IL-1 TRAP, MRA, CTLA4-IG, IL-18 BP, anti-IL-12, Anti-IL1S, BIRB-796, SCIO-469, VX-702, AMG-548, VX-740, Roflumilast, IC-485, CDC-801, S1PI agonists (such as FTY720), a PKC family inhibitor (e.g. Ruboxistaurin or AEB-071) or Mesopram, budesonide, epidermal growth factor, a corticosteroid, cyclosporin, sulfasalazine, an aminosalicylate, 6-mercaptopurine, azathioprine, metronidazole, a lipoxygenase inhibitor, mesalamine, olsalazine, balsalazide, an antioxidant, a thromboxane inhibitor, an IL-1 receptor antagonist, an anti-IL-1 monoclonal antibody, an anti-IL-6 monoclonal antibody, a growth factor, an elastase inhibitor, a pyridinyl-imidazole compound, an antibody to or antagonist of other human cytokines or growth factors (e.g. TNF, LT, IL-1, IL-2, IL-6, IL-7, IL-8, IL-12, IL-15, IL-16, IL-23, EMAP-II, GM-CSF, FGF, and PDGF), a cell surface molecule (e.g. CD2, CD3, CD4, CD8, CD25, CD28, CD30, CD40, CD45, CD69, or CD90 or their ligands), methotrexate, cyclosporine, FK506, rapamycin, mycophenolate mofetil, leflunomide, an NSAID (e.g. ibuprofen), a corticosteroid (e.g. prednisolone), a phosphodiesterase inhibitor, an adenosine agonist, an antithrombotic agent, a complement inhibitor, an adrenergic agent, an agent that interferes with signaling by proinflammatory cytokines such as TNF 5 or IL-1 (e.g. a NIK, IKK, or MAP kinase inhibitor), an IL-1 converting enzyme inhibitor, a TNF converting enzyme inhibitor, a T-cell signaling inhibitor such as kinase inhibitors, a metalloproteinase inhibitor, sulfasalazine, azathioprine, a 6-mercaptopurine, an angiotensin converting enzyme inhibitor, a soluble cytokine receptor (e.g. soluble p55 or p75TNF receptors, siL-1RI, siL-1RII, siL-6R), an anti-inflammatory cytokine (e.g. IL-4, IL-1 0, IL-11, IL-13 or TGF), therapeutic agents that target an intrinsic checkpoint blockade, such as, for example, the gene encoding Cytokine-inducible SH2-containing protein (CISH), antibody BGB-A317, Nivolumab, or Pembrolizumab, atezolizumab, avelumab, durvalumab, ipilimumab, and the like and combinations thereof. The amount of anti-inflammatory agent includes any therapeutically effective amount. For example, in some aspects, the amount of anti-inflammatory agent may be from about 0.01 wt. % to about 5 wt. %, relative to the total amount of the composition, from about 0.1 wt. % to about 1 wt. %, relative to the total amount of the formulation, or any range or individual concentration encompassed by these example ranges.

Depending on the condition being treated, exemplary compositions may contain steroids, antihistamines, sympathomimetics, beta receptor blockers, parasympathomimetics, parasympatholytics, prostaglandins, nonsteroidal anti-inflammatory drugs (NSAIDs), antibiotics, antifungals, topical anesthetics, and combinations thereof.

Further aspects of the disclosure include methods for treating pain by administering a therapeutically effective amount of any of the compositions described or contemplated herein. The step of administering can be carried out by any method including, but not limited to, oral administration, intravenous administration (IV), intramuscular administration (IM), intrathecal administration, subcutaneous administration (SC), sublingual or buccal administration, rectal administration, vaginal administration, cutaneous administration, transdermal administration, and the like and combinations thereof. In some aspects, administering can be carried out by administering orally or by administering topically. In some aspects, the step of administering can be carried out one or two times per hour, one, two, or three times per day, one, two, three, four, or more times per week, and so on depending on the period of exposure or severity of symptoms.

In some aspects, the methods may include the steps of administering any of the exemplary compositions described herein and co-administering one or more of the additional active agents or anti-inflammatory agents described above and the like and combinations thereof. Such additional active agents and anti-inflammatory agents and the like, and combinations thereof, can be administered in separate dosages by any administration route. For example, a composition as described herein can be administered topically, and the additional active agents, anti-inflammatory agents, and combinations thereof may be administered separately topically, orally, and/or by injection.

The compositions and methods of various aspects can be used to treat a patient suffering from various types of pain including, for example, acute pain, chronic pain, neuropathic pain, inflammatory pain, headache pain, somatic pain, visceral pain, and/or referred pain. The term “pain,” as used herein, means any unpleasant sensory experience, usually associated with a physical disorder. The physical disorder may or may not be apparent to a clinician. Pain is of two types: chronic and acute. Acute pain is pain of short duration having a sudden onset. Chronic pain is a pain other than an acute pain. Chronic pain includes neuropathic pain, inflammatory pain, headache pain, somatic pain visceral pain and referred pain.

In some aspects, the compositions of the disclosure can be used to treat pain caused by or otherwise associated with neuropathic pain conditions. Neuropathic pain means abnormal sensory input, resulting in discomfort, from the peripheral nervous system, central nervous systems, or both. Symptoms of neuropathic pain include persistent, spontaneous pain, allodynia (a painful response to a stimulus that normally is not painful), hyperalgesia (an accentuated response to a painful stimulus that usually causes only a mild discomfort, such as a pin prick), and/or hyperpathia (where a short discomfort becomes a prolonged severe pain). Neuropathic pain may be caused, for example, by: a traumatic insult, such as, for example, a nerve compression injury (e.g., a nerve crush, a nerve stretch, a nerve entrapment or an incomplete nerve transection); a spinal cord injury (e.g., a hemisection of the spinal cord); a limb amputation; a contusion; an inflammation (e.g., an inflammation of the spinal cord); a surgical procedure; an ischemic event, including, for example, a stroke and/or a heart attack; exposure to a toxic agent such as, for example, a drug, an alcohol, a heavy metal (e.g., lead, arsenic, mercury), an industrial agent (e.g., a solvent, fumes from a glue) and/or nitrous oxide; a disease such as, for example, an inflammatory disorder, a neoplastic tumor, an acquired immune deficiency syndrome (AIDS), Lyme disease, shingles, varicella-zoster virus infection, a leprosy, a metabolic disease, a peripheral nerve disorder, like neuroma, a mononeuropathy, and/or a polyneuropathy.

Aspects encompass various types of neuropathic pain including, for example, neuralgia (a pain that radiates along the course of one or more specific nerves usually without any demonstrable pathological change in the nerve structure). Types of neuralgia include trigeminal neuralgia, a post-herpetic neuralgia, a postherpetic neuralgia, a glossopharyngeal neuralgia, a sciatica and an atypical facial pain. The various types of neuralgia generally cause short episodes of excruciating pain that can be described in a variety of ways such as “lancinating”, “stabbing,” “sharp,” “like lightning,” “burning,” “like an electric shock” and “itchy.” Neuralgia may occur after infections such as shingles, varicella-zoster virus infection, syphilis, Lyme disease; depression; diabetes; chronic renal insufficiency; porphyria; drug use; and the like and combinations thereof.

In some aspects, exemplary compositions may be used to treat deafferentation. Deafferentation is a loss of the sensory input from a portion of the body, which can be caused by interruption of either peripheral sensory fibers or nerves from the central nervous system. Deafferentation pain syndrome, includes, for example, pain associated with an injury to the brain or spinal cord, post-stroke pain, phantom pain, paraplegia, brachial plexus avulsion injuries, lumbar radiculopathies, and the like and combinations thereof

In some aspects, exemplary compositions may be used to treat complex regional pain syndrome (CRPS). CRPS is a chronic pain syndrome resulting from sympathetically maintained pain and may present in two forms. CRPS 1 is a chronic nerve disorder that occurs most often in the arms or legs after a minor or major injury. CRPS 1 is associated with severe pain; changes in the nails, bone, and skin; and an increased sensitivity to touch in the affected limb. CRPS 2 results from an identified injury to the nerve.

In some aspects, exemplary compositions may be used to treat neuropathy. Neuropathy is a functional or pathological change in a nerve and is characterized clinically by sensory or motor neuron abnormalities. Central neuropathy is a functional or pathological change in the central nervous system. Peripheral neuropathy is a functional or pathological change in one or more peripheral nerves, which relay information from your central nervous system (brain and spinal cord) to muscles and other organs and from your skin, joints, and other organs back to your brain. Risk factors for neuropathy include diabetes, heavy alcohol use, hereditary predisposition, exposure to certain chemicals and drugs, and prolonged pressure on a nerve. Neuropathy can affect any one or a combination of sensory, motor, and autonomic nerves. Symptoms also depend on whether the condition affects the whole body or just one nerve (as from an injury). For example, the methods of the present disclosure may be directed to treating diabetic neuropathic pain (DNP), chemotherapy-induced neuropathic pain (CINP), and the like, or combinations thereof.

Peripheral neuropathies can occur as a result of, for example, hereditary disorders, Charcot-Marie-Tooth disease, Friedreich's ataxia, systemic or metabolic disorders, diabetes (diabetic neuropathy), multiple sclerosis, dietary deficiencies (especially vitamin B-12), excessive alcohol use (alcoholic neuropathy), uremia (from kidney failure), cancer, infectious or inflammatory conditions, HIV/AIDS, hepatitis, Colorado tick fever, diphtheria, Guillain-Barre syndrome. Leprosy, Lyme disease, shingles, varicella-zoster virus infection, polyarteritis nodosa, rheumatoid arthritis, sarcoidosis, Sjogren syndrome, syphilis, systemic lupus erythematosus, amyloid, exposure to toxic compounds, heavy metals (lead, arsenic, mercury, etc.), ischemia (decreased oxygen/decreased blood flow), prolonged exposure to cold temperature, and the like, and combinations thereof.

Polyneuropathy is a peripheral neuropathy involving the loss of movement or sensation to an area caused by damage or destruction to multiple peripheral nerves. Polyneuropathie pain occurs, for example, in post-polio syndrome, postmastectomy syndrome, diabetic neuropathy, alcohol neuropathy, amyloid, toxins, AIDS, hypothyroidism, uremia, vitamin deficiencies, chemotherapy-induced pain, 2′,3′-didexoycytidine (ddC) treatment, Guillain-Barre syndrome, or Fabry's disease.

Mononeuropathy is a peripheral neuropathy involving loss of movement or sensation to an area caused by damage or destruction to a single peripheral nerve or nerve group. Mononeuropathy is most often caused by damage to a local area resulting from injury or trauma, although occasionally systemic disorders may cause isolated nerve damage (as with mononeuritis multiplex). Causes may include trauma, prolonged pressure on the nerve, compression of the nerve by swelling or injury to nearby body structures, and damage causing destruction of the myelin sheath of the nerve or of part of the nerve cell. Examples of mononeuropathic pain include sciatic nerve dysfunction, peroneal nerve dysfunction, radial nerve dysfunction, ulnar nerve dysfunction, cranial mononeuropathy VI, cranial mononeuropathy V, cranial mononeuropathy VII, cranial mononeuropathy III (compression type), cranial mononeuropathy III (diabetic type), axillary nerve dysfunction, carpal tunnel syndrome, femoral nerve dysfunction, tibial nerve dysfunction, Bell's palsy, thoracic outlet syndrome, sixth (abducent) nerve palsy, and the like and combinations thereof.

Generalized peripheral neuropathies are symmetrical, and usually due to various systematic illnesses and disease processes that affect the peripheral nervous system. Generalized peripheral neuropathies include distal axonopathies, which result of some metabolic or toxic derangement of neurons and are often caused by, for example, diabetes, renal failure, deficiency syndromes such as malnutrition and alcoholism, or the effects of toxins or drugs, myelopathies caused by an attack on myelin causing an acute failure of impulse conduction resulting from acute inflammatory demyelinating polyneuropathy (AIDP; aka Guillain-Barre syndrome), chronic inflammatory demyelinating syndrome (CIDP), genetic metabolic disorders (e.g., leukodystrophy), or toxins, neuronopathies that result from destruction of peripheral nervous system (PNS) neurons caused by motor neuron diseases, sensory neuronopathies (e.g., Herpes zoster), toxins, neurotoxins, autonomic dysfunction, or infections and focal entrapment neuropathies such as carpal tunnel syndrome).

In some aspects, exemplary compositions can be used to treat inflammatory pain. For example, exemplary compositions can be used to treat arthritic disorders such as: rheumatoid arthritis; juvenile rheumatoid arthritis; systemic lupus erythematosus (SLE); gouty arthritis; scleroderma; osteoarthritis; psoriatic arthritis; ankylosing spondylitis; Reiter's syndrome (reactive arthritis); adult Still's disease; arthritis from a viral infection; arthritis from a bacterial infection, such as, a gonococcal arthritis and a non-gonococcal bacterial arthritis (septic arthritis); tertiary Lyme disease; tuberculous arthritis; arthritis from fungal infection, such as, blastomycosis, and the like and combinations thereof. In some aspects, exemplary compositions can be used to treat autoimmune diseases such as, for example, Guillain-Barre syndrome, Hashimoto's thyroiditis, pernicious anemia, Addison's disease, type I diabetes, systemic lupus erythematosus, dermatomyositis, Sjogren's syndrome, lupus erythematosus, multiple sclerosis, myasthenia gravis, Reiter's syndrome, Grave's disease, Morgellons disease, and the like and combinations thereof. In some aspects, exemplary compositions may be used to treat connective tissue disorders such as, for example, spondylarthrites, dermatomyositis, fibromyalgia, and the like and combinations thereof. In some aspects, exemplary compositions can be used to treat injuries and inflammation caused by injury such as, for example, crushing, punctures, stretches of joints or tissues associated with joints. In some aspects, exemplary compositions can be used to treat neuritis, an inflammatory process affecting a nerve or group of nerves with symptoms including pain, paresthesia, paresis, or hypesthesia (numbness). Examples of neuritis include brachial neuritis, retrobulbar neuropathy, optic neuropathy, vestibular neuritis, and the like and combinations thereof.

In some aspects, exemplary compositions may be used to treat headache pain including, for example, muscular/myogenic headache, tension headache, episodic tension headache, chronic tension headache, vascular headache, migraine headache including, migraine without aura (common migraine), migraine with aura (classic migraine), menstrual migraine, migraine equivalent (acephalic headache), complicated migraine, abdominal migraine, and mixed tension migraine, cluster headaches, high blood pressure headache, traction and inflammatory headache, hormone headache, rebound headache, chronic sinusitis headache resulting from, for example, bacterial infection, fungal infection, viral infection, allergies, or autoimmune disease of the paranasal sinuses, and the like, organic headache, ictal headaches, and the like and combinations thereof.

In some aspects, exemplary compounds may be used to treat pain caused by or otherwise associated with somatic pain conditions such as, for example, excessive muscle tension, sprains, strains, repetitive motion disorders resulting from, for example, overuse of the hands, wrists, elbows, shoulders, neck, back, hips, knees, feet, legs, or ankles, muscle disorders resulting from, for example, polymyositis, dermatomyositis, lupus, fibromyalgia, polymyalgia rheumatica, and rhabdomyolysis, myalgia, infections including, for example, abscesses in muscle, trichinosis, influenza, Lyme disease, malaria, Rocky Mountain spotted fever, avian influenza, common cold, community-acquired pneumonia, meningitis, monkeypox, severe acute respiratory syndrome, toxic shock syndrome, trichinosis, typhoid fever, upper respiratory tract infection, and the like, drugs including, for example, cocaine, statins for lowering cholesterol (such as atorvastatin, simvastatin, and lovastatin), ACE inhibitors for lowering blood pressure (such as enalapril and captopril), and the like, and combinations thereof.

In some aspects, exemplary compositions can be used to treat visceral pain originating from body's viscera or organs including, for example, functional visceral pain such as pain associated with irritable bowel syndrome, chronic functional abdominal pain (CFAP), functional constipation, functional dyspepsia, non-cardiac chest pain (NCCP), and a chronic abdominal pain, chronic gastrointestinal inflammation such as gastritis, Crohn's disease, ulcerative colitis, microscopic colitis, diverticulitis, gastroenteritis, interstitial cystitis, intestinal ischemia, cholecystitis, appendicitis, gastroesophageal reflux, ulcer, nephrolithiasis, urinary tract infection, pancreatitis, hernia, and the like, autoimmune pain such as sarcoidosis and vasculitis, organic visceral pain such as pain resulting from a traumatic, inflammatory, or degenerative lesion of the gut or produced by a tumor impinging on sensory innervation, treatment-induced visceral pain such as pain attendant to chemotherapy therapy or radiation therapy, and the like and combinations thereof.

In some aspects, exemplary compositions can be used to treat pain caused by or otherwise associated with referred pain conditions such as, for example, pain associated with intervertebral disc herniation, compressed nerves in, for example, the thigh, knee, or foot, myocardial ischemia, and the like and combinations thereof.

In any embodiment described herein, the compositions and methods of the present disclosure are directed to treating trigeminal neuralgia and reducing and/or managing pain relating thereto. In some embodiments, there is provided a method of treating trigeminal neuralgia which includes administering to a patient in need thereof a therapeutically effective amount of Trimeprazine or a pharmaceutically acceptable salt thereof, thereby treating the trigeminal neuralgia in the patient.

In some embodiments, the therapeutically effective amount of Trimeprazine or a pharmaceutically acceptable salt thereof is administered once a day. In some embodiments, the therapeutically effective amount of Trimeprazine or a pharmaceutically acceptable salt thereof Trimeprazine or a pharmaceutically acceptable salt thereof is administered twice a day. The therapeutically effective amount of Trimeprazine or a pharmaceutically acceptable salt thereof may be administered by any method as described herein, such as oral administration, intravenous administration (IV), intramuscular administration (IM), intrathecal administration, subcutaneous administration (SC), sublingual or buccal administration, rectal administration, vaginal administration, cutaneous administration, transdermal administration, or combinations thereof. In some embodiments, the therapeutically effective amount of Trimeprazine or a pharmaceutically acceptable salt thereof is administered orally. In some embodiments, the therapeutically effective amount of Trimeprazine or a pharmaceutically acceptable salt thereof is administered sublingually.

In some embodiments, the therapeutically effective amount of Trimeprazine or a pharmaceutically acceptable salt thereof is about 0.5 mg to about 20 mg, such as about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, or any range or value contained therein. In some embodiments, the therapeutically effective amount of Trimeprazine or a pharmaceutically acceptable salt thereof is about 0.05 mg/kg to about 5 mg/kg, such as about 0.05 mg/kg, about 0.06 mg/kg, about 0.07 mg/kg, about 0.08 mg/kg, about 0.09 mg/kg, about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, or any range or value contained therein.

The trigeminal neuralgia may be Type 1 or Type 2, or combinations thereof. Type 1 TN may present as intense, brief, sudden, and/or blazing facial which may last from seconds to minutes. Type 2 TN may present as a persistent stinging, aching, or burning soreness and may be relatively less intense than Type 1. Patients may experience both Type 1 and Type 2 TN, simultaneously or periodically. Further classifications of TN include primary, which results from vascular compression of nerves; secondary, which often results from neurologic disease such as multiple sclerosis or a tumor or cyst near the trigeminal nerve; and idiopathic, wherein no cause has been identified.

In some embodiments, treating the trigeminal neuralgia includes reducing patients' pain. Reduction in pain may be evaluated by any method known to those skilled in the art, including pain assessment scales such as OPQRST, QISS TAPED, SOCRATES, Pain Risk Factors Assessment Form, Numerical Rating Scale, Visual Analog Scale, Penn Facial Pain Scale (Penn-FPS), Penn Facial Pain Scale Revised (Penn-FPS-R), Brief Pain Inventory-Short Form (BPI-SF), Brief Pain Inventory Pain Interference Index (BPI-PII), Patient Global Impression of Change (PGIC), Faces Pain Scale-Revised (FPS-R), Burchiel Questionnaire, McGill Pain Questionnaire, Barrow Neurological Institute (BNI) pain intensity score, and other patient-reported outcome (PRO) and clinician-delivered assessments familiar to those skilled in the art. Other assessments which evaluate the impact of pain and the patient's ability to complete daily activities may also be employed, such as EuroQoL 5-Dimension 5 Level (EQ-5D-5L), Work Productivity and Activity Impairment (WPAI), and the like. Treating the trigeminal neuralgia in the patient may include improving and/or preventing an increase in one or more of any of the assessments as described herein.

In some embodiments, reducing the patients' pain includes an improvement in the patients' pain as measured by Penn-FPS-R score, Penn-FPS score, or combinations thereof. In some embodiments, reducing the patients' pain includes preventing an increase in patients' pain as measured by Penn-FPS-R score, Penn-FPS score, or combinations thereof.

In some embodiments, treating the trigeminal neuralgia includes an improvement in the patient's Penn-FPS-R score, Penn-FPS score, PGIC score, EQ-5D-5L score, WPAI score, or combinations thereof. In some embodiments, treating the trigeminal neuralgia includes preventing an increase in the patient's Penn-FPS-R score, PGIC score, Penn-FPS score, EQ-5D-5L score, BNI Pain Intensity score or WPAI score, preventing a low PGIC score, or combinations thereof.

The Penn-FPS-R scale asks patients to circle a number that best describes how much their pain interferes with items including eating a meal, touching the face (including moving stray hairs, hugging, kissing, itching), brushing or flossing teeth, smiling or laughing, talking, opening the mouth widely, biting or chewing, self-care (including washing face or hair, shaving, applying makeup), activities with temperature change (including moving outside or between air-conditioned rooms), daily activities (including work, exercise, housework), mood (the way the patient is feeling), and relationships (with friends, family, partners, etc.). Endpoints for treating TN include reduction in the overall severity and duration of facial pain, along with reduction in frequency and severity of paraxosyms (intense flashes of pain related to TN) and reduction in use of existing standard of care pain medications for TN, including elimination of use of existing standard of care pain medications for TN.

In some embodiments, there is embodied a method for the treatment of the pain associated with trigeminal neuralgia comprising administering to a patient in need thereof a therapeutically effective amount of Trimeprazine or a pharmaceutically acceptable salt thereof, thereby treating the trigeminal neuralgia in the patient.

In some embodiments, the therapeutically effective amount of Trimeprazine or a pharmaceutically acceptable salt thereof is administered once a day.

In some embodiments, the therapeutically effective amount of Trimeprazine or a pharmaceutically acceptable salt thereof is administered twice a day.

In some embodiments, the therapeutically effective amount of Trimeprazine or a pharmaceutically acceptable salt thereof is administered via oral administration, intravenous administration (IV), intramuscular administration (IM), intrathecal administration, subcutaneous administration (SC), sublingual or buccal administration, rectal administration, vaginal administration, cutaneous administration, transdermal administration, nasal administration, or combinations thereof.

In some embodiments, the therapeutically effective amount of Trimeprazine or a pharmaceutically acceptable salt thereof is administered via oral administration.

In some embodiments, the therapeutically effective amount of Trimeprazine or a pharmaceutically acceptable salt thereof is administered via an oral thin film formulation.

In some embodiments, the therapeutically effective amount of Trimeprazine or a pharmaceutically acceptable salt thereof is about 0.5 mg to about 20 mg.

In some embodiments, the therapeutically effective amount of Trimeprazine or a pharmaceutically acceptable salt thereof is about 2.5 to about 5 mg.

In some embodiments, the therapeutically effective amount of Trimeprazine or a pharmaceutically acceptable salt thereof is about 0.5 to about 2.5 mg.

In some embodiments, the treatment of the pain associated with trigeminal neuralgia comprises a reduction in the frequency of pain, a reduction in the duration of pain, a reduction in the intensity of pain, or any combination thereof.

In some embodiments, a reduction in the duration of pain comprises a reduction in the duration of a single episode of pain, a reduction in the duration of a series of episodes of pain, and any combination thereof.

In some embodiments, a series of episodes of pain comprises at least two separate episodes of pain.

In some embodiments, the pain associated with trigeminal neuralgia is stabbing pain, lancinating pain, shock-like pain, electric shock-like pain, constant aching, burning, stabbing pain of somewhat lower intensity than Type 1 TN, and any combination thereof.

In some embodiments, a reduction in the frequency of pain, a reduction in the duration of pain, a reduction in the intensity of pain, or any combination thereof is measured using a patient diary, a PGIC score, a MSQ score, a Penn-FPS-R score, a Penn-FPS score, an EQ-5D-5L score, Barrow Neurological Institute pain intensity score, a WPAI score or any combination thereof.

In some embodiments, administering the therapeutically effective amount of Trimeprazine or a pharmaceutically acceptable salt thereof comprises administering an initial dose of between 2.5 to about 5.0 mg.

In some embodiments, administering the therapeutically effective amount of Trimeprazine or a pharmaceutically acceptable salt thereof comprises administering an initial dose of between about 0.5 mg to about 2.5 mg, such as about 0.5 mg, about 1 mg, about 1.5 mg, about 2 mg, about 2.5 mg, or any value contained within a range formed by any two of the preceding values.

Some embodiments further comprise administering an additional therapeutically effective amount of Trimeprazine or a pharmaceutically acceptable salt thereof, about every 30 minutes to about every 5 hours following the initial dose if treatment of pain associated with trigeminal neuralgia is not achieved.

In some embodiments, the additional, additional therapeutically effective amount of Trimeprazine or a pharmaceutically acceptable salt thereof is increased about every 30 minutes to about every 5 hours until treatment of the pain associated with trigeminal neuralgia is achieved. For example, the initial dose may be about 0.5 mg, followed by administering a dose of about 1 mg about 30 minutes later. In some embodiments, the initial dose may be about 1 mg, followed by administering a dose of about 2.5 mg about 2 hours later. Various combinations of an initial dose of about 0.5 to about 2.5 mg followed by an additional dose (which is larger than the initial dose) about 30 minutes to about 5 hours after the initial dose is administered are contemplated.

In some embodiments, once treatment of pain associated with trigeminal neuralgia is achieved, the additional therapeutically effective amount of Trimeprazine or a pharmaceutically acceptable salt thereof is maintained as the minimum effective level used for the treatment of pain associated with trigeminal neuralgia.

In some embodiments, the minimum effective level of therapeutically effective amount of Trimeprazine or a pharmaceutically acceptable salt thereof is administered until treatment of pain treatment of pain associated with trigeminal neuralgia is no longer achieved, after which the minimum effective level of therapeutically effective amount of Trimeprazine or a pharmaceutically acceptable salt thereof may be further increased until treatment of pain treatment of pain associated with trigeminal neuralgia is once again achieved.

In some embodiments, at least about every 3 months, the therapeutically effective amount of Trimeprazine or a pharmaceutically acceptable salt thereof administered is decreased to the minimum effective level of therapeutically effective amount of Trimeprazine or a pharmaceutically acceptable salt thereof.

In some embodiments, at least about every 3 months, administration of the therapeutically effective amount of Trimeprazine or a pharmaceutically acceptable salt thereof administered is discontinued.

There is provided a method of reducing a patient's pain from trigeminal neuralgia which includes administering to the patient in need thereof a therapeutically effective amount of Trimeprazine or a pharmaceutically acceptable salt thereof, thereby reducing the patient's pain. The method of reducing a patient's pain from trigeminal neuralgia may use any of the compositions, formulations, or dosing regimens of any embodiment described herein.

In some embodiments, reducing the patient's pain includes an improvement in the patient's pain as measured by Penn-FPS score compared to the patient's pain as measured by Penn-FPS score prior to treatment. In some embodiments, reducing the patient's pain includes preventing an increase in the patient's pain as measured by Penn-FPS score compared to the patient's pain as measured by Penn-FPS score prior to treatment.

In some embodiments, reducing the patient's pain includes an improvement in the patient's pain as measured by Penn-FPS-R score compared to the patient's pain as measured by Penn-FPS-R score prior to treatment. In some embodiments, reducing the patient's pain includes comprises preventing an increase in the patient's pain as measured by Penn-FPS-R score compared to the patient's pain as measured by Penn-FPS-R score prior to treatment.

In some embodiments, reducing the patient's pain includes an improvement in the patient's PGIC score at the end of the treatment period. In some embodiments, reducing the patient's pain includes preventing a low PGIC score at the end of the treatment period.

The self-report measure Patient Global Impression of Change (PGIC) reflects a patient's belief about the efficacy of treatment. PGIC is a 7 point scale depicting a patient's rating of overall improvement. Patients rate their change as “very much improved,” “much improved,” “minimally improved,” “no change,” “minimally worse,” “much worse,” or “very much worse.” As used herein, a low PGIC score is considered to be a score of “minimally worse,” “much worse,” or “very much worse” at the end of the treatment period. Accordingly, the present methods of treating trigeminal neuralgia and reducing pain related thereto include preventing scores of “minimally worse,” “much worse,” or “very much worse” at the end of the treatment period. An improvement in the patient's PGIC score at the end of the treatment period includes a score of “very much improved,” “much improved,” or “minimally improved” at the end of the treatment period.

There is provided a method for treating pain in a patient which includes administering to the patient a therapeutically effective amount of Trimeprazine or a pharmaceutically acceptable salt thereof, thereby treating the patient's pain. In some embodiments, the therapeutically effective amount of Trimeprazine or a pharmaceutically acceptable salt thereof is about 0.5 mg to about 80 mg, such as about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg, about 50 mg, about 55 mg, about 60 mg, about 70 mg, about 75 mg, about 80 mg, or any range or value contained therein.

In some embodiments, the pain may be caused by any acute, chronic, neuropathic, or other pain source as disclosed herein. For example, in some embodiments, the pain may be caused by trigeminal neuralgia, post-herpetic neuralgia, postherpetic neuralgia, neuritis, brachial neuritis, retrobulbar neuropathy, optic neuropathy, vestibular neuritis, glossopharyngeal neuralgia, sciatica atypical facial pain, deafferentation pain syndrome, complex regional pain syndrome (CRPS), central neuropathy, peripheral neuropathy, diabetic neuropathic pain (DNP), chemotherapy-induced neuropathic pain (CINP), polyneuropathy, mononeuropathy, generalized peripheral neuropathies, or combinations thereof.

In some embodiments, the pain may be caused by arthritic disorders such as: rheumatoid arthritis; juvenile rheumatoid arthritis; systemic lupus erythematosus (SLE); gouty arthritis; scleroderma; osteoarthritis; psoriatic arthritis; ankylosing spondylitis; Reiter's syndrome (reactive arthritis); adult Still's disease; arthritis from a viral infection; arthritis from a bacterial infection, such as, a gonococcal arthritis and a non-gonococcal bacterial arthritis (septic arthritis); tertiary Lyme disease; tuberculous arthritis; arthritis from fungal infection, such as, blastomycosis, or combinations thereof.

In some embodiments, the pain may be caused by autoimmune diseases such as, for example, Guillain-Barre syndrome, Hashimoto's thyroiditis, pernicious anemia, Addison's disease, type I diabetes, systemic lupus erythematosus, dermatomyositis, Sjogren's syndrome, lupus erythematosus, multiple sclerosis, myasthenia gravis, Reiter's syndrome, Grave's disease, Morgellons disease, or combinations thereof.

In some embodiments, the pain may be caused by connective tissue disorders such as, for example, spondylarthrites, dermatomyositis, fibromyalgia; injuries and inflammation caused by injury such as, for example, crushing, punctures, stretches of joints or tissues associated with joints, or combinations thereof.

In some embodiments, the pain may be caused by muscular/myogenic headache, tension headache, episodic tension headache, chronic tension headache, vascular headache, migraine headache including, migraine without aura (common migraine), migraine with aura (classic migraine), menstrual migraine, migraine equivalent (acephalic headache), complicated migraine, abdominal migraine, and mixed tension migraine, cluster headaches, high blood pressure headache, traction and inflammatory headache, hormone headache, rebound headache, chronic sinusitis headache resulting from, for example, bacterial infection, fungal infection, viral infection, allergies, or autoimmune disease of the paranasal sinuses, or combinations thereof.

In some embodiments, the pain may be caused by somatic pain conditions such as, for example, excessive muscle tension, sprains, strains, repetitive motion disorders resulting from, for example, overuse of the hands, wrists, elbows, shoulders, neck, back, hips, knees, feet, legs, or ankles or combinations thereof.

In some embodiments, the pain may be caused by muscle disorders resulting from, for example, polymyositis, dermatomyositis, lupus, fibromyalgia, polymyalgia rheumatica, and rhabdomyolysis, myalgia, infections including, for example, abscesses in muscle, trichinosis, influenza, COVID-19, Lyme disease, malaria, Rocky Mountain spotted fever, avian influenza, common cold, community-acquired pneumonia, meningitis, monkeypox, severe acute respiratory syndrome, toxic shock syndrome, trichinosis, typhoid fever, upper respiratory tract infection, or combinations thereof.

In some embodiments, the pain may be caused by visceral pain originating from body's viscera or organs including, for example, functional visceral pain such as pain associated with irritable bowel syndrome, chronic functional abdominal pain (CFAP), functional constipation, functional dyspepsia, non-cardiac chest pain (NCCP), and a chronic abdominal pain, chronic gastrointestinal inflammation such as gastritis, Crohn's disease, ulcerative colitis, microscopic colitis, diverticulitis, gastroenteritis, interstitial cystitis, intestinal ischemia, cholecystitis, appendicitis, gastroesophageal reflux, ulcer, nephrolithiasis, urinary tract infection, pancreatitis, hernia, and the like and combinations thereof.

In some embodiments, the pain may be caused by autoimmune pain such as sarcoidosis and vasculitis, organic visceral pain such as pain resulting from a traumatic, inflammatory, or degenerative lesion of the gut or produced by a tumor impinging on sensory innervation, treatment-induced visceral pain such as pain attendant to chemotherapy therapy or radiation therapy, and the like and combinations thereof.

In some embodiments, the pain may be caused by referred pain conditions such as, for example, pain associated with intervertebral disc herniation, compressed nerves in, for example, the thigh, knee, or foot, myocardial ischemia, and the like and combinations thereof.

In any of these embodiments, reducing the patient's pain may include reducing and/or preventing an increase in the patient's pain as assessed by patient-reported outcomes (PRO) or clinician-delivered assessments familiar to those skilled in the art. The assessment utilized to evaluate pain is not particularly limited and may be any assessment disclosed herein, or any assessment known to those skilled in the art, including but not limited to assessments relevant to the conditions and sources of pain disclosed herein.

As described herein, the methods of the present disclosure may replace existing standard of care treatments for trigeminal neuralgia or any acute pain, chronic pain, neuropathic pain, inflammatory pain, headache pain, somatic pain, visceral pain, and/or referred pain as disclosed herein, or may be used in addition to such treatments in order to provide further relief of symptoms as described herein. Without wishing to be bound by theory, examples of existing standard of care treatments are provided herein.

Sources of pain from any condition as described herein may be treated by a variety of medications. For example, medications for conditions as described herein and pain relating thereto may include anticonvulsants such as Gabapentin and Pregabalin; tricyclic antidepressants such as Amitriptyline and Nortriptyline; serotonin-norepinephrine reuptake inhibitors such as Duloxetine, Venlafaxine, and Desvenlafaxine; Opioid-like Medications such as Tramadol and Tapentadol ER; opioids such as oxycodone, hydrocodone, tramadol, codeine, and morphine; topical medications such as Lidocaine patches, Capsaicin creams and patches, and isosorbide dinitrate spray, compounded gel containing baclofen, amitriptyline, and ketamine; Selective Serotonin Reuptake Inhibitors (SSRIs) such as Citalopram, Paroxetine, and Escitalopram; non-steroidal anti-inflammatory drugs (NSAIDs) such as Aspirin, Ibuprofen Naproxen Sodium, N-methyl-D-aspartate (NMDA) receptor agonists such as ketamine, dextromethorphan, memantine, and amantadine, as well as opioids such as methadone, dextropropoxyphene, and ketobemidone which are also antagonists at the NMDA receptor; Acetaminophen; nerve stimulation including neuro stimulation, either local or spinal; psychological support such as cognitive behavioral therapy; steroid shots including Betamethasone; and local anesthetics such as lidocaine and bupivacaine hydrochloride. Other medications for the conditions described herein may also be used, as would be familiar to one of ordinary skill in the art. The present methods and compositions may replace or be used in combination with the other medications disclosed herein.

Additional examples of other medications and exemplary dosing regimens which may be used to treat the conditions disclosed herein are also described. Without wishing to be bound by theory, the medications disclosed herein may be used to treat various pain conditions (such as those disclosed herein) and may be replaced by or used in combination with the compositions and methods of the present disclosure to effectively treat various sources of pain.

Carbamazepine may be administered at an initial dose of about 200 mg, for example two to four times per day. The dose range may be about 200 mg to about 1200 mg, with titration at about 200 mg every 3 days. Tapering may be 200 mg every 7 days. Potential side effects include dizziness, drowsiness, fatigue, ataxia, diplopia, nausea, cognitive slowing, hyponatraemia leucopenia, thrombocytopenia, skin reactions, and abnormal liver function tests. Carbamazepine may be used to treat conditions including but not limited to trigeminal neuralgia. Carbamazepine is approved in the EU for trigeminal neuralgia and diabetic peripheral neuropathic pain and approved in the US for trigeminal neuralgia. Carbamazepine stabilizes membranes at voltage-gated sodium channels on sensitized nociceptive neurons in PNS and CNS, and reduces spontaneous activity of these neurons.

Oxcarbazepine may be administered at an initial dose of about 300 mg, for example four times per day. The dose range may be about 300 mg to about 1800 mg, with titration at about 300 mg every 3 days. Tapering may be 300 mg every 7 days. Potential side effects include dizziness, drowsiness, fatigue, nausea, ataxia, hyponatraemia, and skin reaction. Oxcarbazepine may be used to treat conditions including but not limited to trigeminal neuralgia.

Lamotrigine may be administered at an initial dose of about 25 mg, for example two times per day. The dose range may be about 25 mg to about 400 mg, with titration at about 25 mg for 2 weeks, 50 mg for one week, then increase the dose by about 50 mg every week. Tapering may be 50 mg every 7 days. Potential side effects include dizziness, drowsiness, fatigue, headache, gastrointestinal symptoms, irritability, sleep disorders, tremor, cognitive slowing, and rash. Lamotrigine may be used to treat conditions including but not limited to trigeminal neuralgia.

Gabapentin may be administered at an initial dose of about 300 mg, for example three times per day. The dose range may be about 300 mg to about 3600 mg, with titration at about 300 mg every 3 days. Tapering may be 300 mg every 7 days. Potential side effects include dizziness, confusion, fatigue, ataxia, somnolence, suicidal behavior, withdrawal-precipitated seizure frequency, multi-organ hypersensitivity, peripheral edema, ataxia or gait disturbance, diarrhea, increased risk of infection, gastrointestinal symptoms, and weight gain; use cautiously with opioids. Gabapentin is an anticonvulsant/anti-epileptic and is approved by the FDA for the treatment of PHN in adults and may also be used in the treatment of conditions including but not limited to trigeminal neuralgia and DNP. Gabapentin is approved in the EU for peripheral neuropathic pain. It is structurally related to the GABA neurotransmitter and works by binding to the α2-δ site of voltage-gated calcium channels, which reduces the release of excitatory neurotransmitters.

Pregabalin may be administered at an initial dose of about 150 mg, for example two times per day. The dose range may be about 150 mg to about 600 mg, with titration at about 150 mg every 3 days. Tapering may be 100 mg every 7 days. Potential side effects include dizziness, confusion, ataxia, increased risk of infection, gastrointestinal symptoms, and weight gain. Pregabalin is an FDA-approved treatment for post-herpetic neuralgia (PHN) and may also be used in the treatment of conditions including but not limited to trigeminal neuralgia. Pregabalin is also approved in the US for diabetic peripheral neuropathic pain and neuropathic pain associated with spinal cord injury, and in the EU for peripheral and central neuropathic pain. It acts similarly to gabapentin by binding to calcium channels and influencing neurotransmitter release, but is more potent and used at lower doses. Adverse effects include dizziness, water retention, visual disturbances, somnolence, ataxia, euphoria, and vertigo. A study in Japan reported that pregabalin's analgesic property is 6 times that of gabapentin in PHN. Dose increases should be done gradually and carefully to prevent adverse effects despite pain reduction.

Baclofen may be administered at an initial dose of about 15 mg, for example three times per day. The dose range may be about 15 mg to about 90 mg, with titration at about 15 mg every 3 days. Tapering may be 15 mg every 7 days. Potential side effects include confusion, dizziness, drowsiness, gastrointestinal symptoms, euphoria, and hallucinations. Baclofen may be used to treat conditions including but not limited to trigeminal neuralgia. Baclofen may act by a combined mode of action, i.e., GABAergic modulation, blockade of sodium channels and glutamatergic (NMDA) receptors.

Botulinum toxin type A may be administered at an initial dose of about 25-195 units, for example every 12 weeks. The dose range may be about 25 units to about 195 units. Potential side effects include transient facial asymmetry, transient bruising at injection site, transient drooling and difficulty chewing. Botulinum toxin type A may be used to treat conditions including but not limited to trigeminal neuralgia. Botulinum Toxin A (BTX-A) is a toxin produced by Clostridium botulinum and has been used for treating various diseases, including dystonia, spasticity, brain paralysis, strabismus, and chronic pain of different origins, including PHN. Its mechanism of action is not fully understood. It mainly acts by inhibiting the release of pain mediators from the nerve terminals and dorsal root ganglions, reducing inflammation around the nerve endings, deactivating sodium channels, and exhibiting axonal transport. BTX-A was found to be effective in reducing pain in 18 (31%) cases and showed significant outcomes in 27 (46.6%) cases of PHN symptoms in a study conducted on 58 patients. The side effects of BTX-A treatment were mild and included pain at the injection site, which disappeared within a week without any treatment.

Duloxetine is a selective serotonin and norepinephrine reuptake inhibitor (SNRI) that increases the levels of these neurotransmitters in the brain and spinal cord, which helps to modulate pain transmission. Duloxetine is started at 30 mg daily and titrated up to a maximum dose of 60 mg/day. A representative dose may be about 60 mg to about 120 mg per day. Adverse effects may include nausea, dry mouth, dizziness, fatigue, constipation, Xerostomia, decreased appetite, somnolence, sweating, gastrointestinal discomfort, and insomnia. Duloxetine may be used to treat conditions including but not limited to PHN, DNP, and CIPN. Duloxetine is a potent inhibitor of neuronal serotonin and norepinephrine reuptake and a less potent inhibitor of dopamine reuptake. Duloxetine has no significant affinity for dopaminergic, adrenergic, cholinergic, histaminergic, opioid, glutamate, and GABA receptors. Also related to duloxetine's action at the spinal cord is its modulation of pain. Increasing the concentration of serotonin and norepinephrine in the dorsal horn of the spinal cord increases descending inhibition of pain through activation of 5-HT_{1A}, 5-HT_{1B}, 5-HT_{1D}, 5-HT₂, 5-HT₃, α₁-adrenergic, and α₂-adrenergic receptors.

Venlafaxine may be used in the treatment of conditions including but not limited to DNP and CIPN. A representative dose may be about 75 mg to about 225 mg per day. Potential side effects include somnolence, dizziness, and mild gastrointestinal problems. The exact mechanism of action of venlafaxine in the treatment of various psychiatric conditions has not been fully elucidated; however, it is understood that venlafaxine and its active metabolite O-desmethylvenlafaxine (ODV) potently and selectively inhibits the reuptake of both serotonin and norepinephrine at the presynaptic terminal.

Lidocaine is a local anesthetic used topically to provide surface analgesia for chronic pain conditions including but not limited to PHN and DNP. It is formulated as a plaster containing 5% lidocaine that is applied to the undamaged skin once daily for 12 hours with a plaster-free interval of 12 hours. Maximum 3 lidocaine plasters at 5% can be applied to intact skin once daily for a period of 12 hours. Lidocaine may act as a mechanical barrier to the area of allodynia, preventing stimulation. Lidocaine is released continuously after application and only 3% of the drug reaches the systemic circulation, which is well below toxic concentrations. Lidocaine is extensively metabolized by the liver and excreted by the kidney. The adverse effects observed after lidocaine plaster treatment are local skin reactions such as pruritus, erythema, rash, burning sensation, and edema. Lidocaine is well-tolerated by individuals of any age with minimal adverse effects and is better tolerated than systemic treatment with pregabalin. Lidocaine is approved in the EU and US for PHN. Lidocaine works by partially inhibiting the voltage-gated calcium channels (blocking of abnormally functioning (sensitized) Nav1.7 and Nav1.8 sodium channels in the dermal nociceptors), reducing the discharge of ectopic activity in damaged afferent pain receptors, and may have anti-inflammatory properties via regulation of T cell activity and suppression of nitric oxide production.

Capsaicin is a selective agonist of TRPV1 channels found in nociceptors in the skin, and exposure to capsaicin activates TRPV1 which causes an influx of calcium and also inhibits the electron-chain transport resulting in a loss of cellular integrity and defunctionalization of nociceptor nerve fibers for a prolonged period, leading to pain relief. High concentration 8% capsaicin transdermal patches are used for neuropathic pain treatment, requiring a single application for up to 3 months. The patch contains 8% capsaicin (640 mcg/cm2), and a total of 179 mg of capsaicin in a single patch. Adverse effects like burning, erythema, pain, dryness, edema, and pruritus can be managed with topical analgesics such as lidocaine. Capsaicin cream may also be used, for example at 0.075% four times per day. Skin-site side reactions may occur. Capsaicin may be used for the treatment of conditions including but not limited to DNP. In the EU, capsaicin is approved for topical treatment of peripheral neuropathic pain as monotherapy or in combination with other pharmaceutical products for the treatment of pain. In the US, capsaicin is approved for postherpetic neuralgia (PHN) and diabetic peripheral neuropathic (DNP) pain. A cream formulation of capsaicin may selectively activate TRPM8, which is also activated upon sensation of cold temperature and after sensory nerve injury.

Clonidine gel may be used for the treatment of conditions including but not limited to DNP. Single doses of 0.65 g of gel may be applied three times daily. Skin-site reactions may occur. Topical clonidine, is a presynaptic α-2 adrenergic receptor agonist with antinociceptive activity, was associated with pain relief in DNP in a small number of studies of low-to-moderate quality.

α-Lipoic acid gel may be used for the treatment of conditions including but not limited to DNP. A representative dose may be about 600-1800 mg orally or 600 mg/day intravenously for 3 weeks, excluding weekends. Potential side effects include nausea, vomiting, abdominal discomfort, and diarrhea. α-Lipoic acid is a natural thiol with potent antioxidant properties and is used as a dietary supplement.

Opioids, such as oxycodone, hydrocodone and morphine have good analgesic effects; however, their use in PHN is controversial due to concerns about addiction and dependence. Opioids modulate pain by interacting with various opioid receptors of the mu, kappa, and delta classes that are present both centrally and peripherally during an inflammatory response. These receptors coupled with inhibitory G-proteins, when activated, causes closure of voltage-gated calcium channels leading to potassium efflux and hyperpolarization, and decreases the production of cyclic adenosine monophosphate. These mechanisms result in a reduction of neuronal cell excitability and transmission of nociceptive impulses, thereby altering the response to pain. Several clinical studies have demonstrated the effectiveness of opioids in managing neuropathic pain, including PHN. Adverse effects of opioids include nausea, pruritus, drowsiness, constipation, and sedation, and should be cautiously used in patients with a history of substance abuse. Opioids are typically used as second or third-line agents, and at a lower dose as adjunct therapy to provide immediate pain relief while titrating other first-line agents to reach their therapeutic dose. Maximum dosage of oxycodone may be about 120 mg per day divided into two doses. Chronic use may lead to tolerance, frequent dose escalation, and hyperalgesia. Oxycodone and its active metabolites can selectively bind to the mu opioid receptor, but also the kappa and delta opioid receptors in the central nervous system and periphery and induce a G protein coupled receptor signaling pathway. Activation of mu opioid receptors inhibits N-type voltage operated calcium channels, inhibiting responses to pain. Some opioids also act via noradrenergic and serotonergic reuptake inhibition on the inhibitory system of descending nerves (pain inhibition). Opioids are approved in the US and EU for moderate-to-severe pain.

Cannabinoids may be used for the off-label treatment of pain as described herein. Cannabinoids are agonists at CB1 receptors in CNS, spinal cord, and peripheral nerves, and may act via inhibition of neuronal excitability. Some cannabinoid compounds are psychoactive, and synthetic cannabinoid receptor agonists may have higher psychosis-inducing potential than natural cannabis and should be considered with care.

Tapentadol may be used for the treatment of conditions including but not limited to DNP. A representative dose may be about 100 mg to about 500 mg per day. Potential side effects include dizziness, somnolence, headache, fatigue, and gastrointestinal problems. Tapentadol is a centrally acting synthetic analgesic that is 18 times less potent than morphine in binding mu-opioid receptors. It also increases norepinephrine concentrations in the brains of rats via inhibition of norepinephrine reuptake.

Tramadol is a weak opioid that acts on the mu receptor and inhibits serotonin and norepinephrine reuptake. The drug has a maximum daily dose of 400 mg and is titrated gradually by 50-100 mg. Tramadol is considered a mild opioid and has proven to be less effective in pain relief in PHN than other opioids, but it is better tolerated and a safer alternative. It may be a better option for patients with a history of substance abuse or cardiac problems. Adverse effects include nausea, somnolence, constipation, dizziness, headache, vertigo, and increased risk of seizures in higher doses. Caution is recommended when using tramadol in patients with a history of seizures or those taking drugs to reduce the seizure threshold.

Other drugs may also be used as standard of care treatments for TN or other conditions resulting in pain as described herein. For example, Pimozide is a dopamine receptor antagonist, is used mainly in the management of Tourette syndrome. It was found effective in a randomized, double-blind crossover trial of 48 patients with refractory TN.

Topiramate is another drug which may be used to treat TN. The exact mechanism of action of topiramate is unknown. However, its pain-modulating effect might be related to its property of blockage of the voltage-gated sodium channel and an augmentation of GABA activity by binding to a non-benzodiazepine site on the GABAA receptor. Topiramate (100-400 mg/d) was found effective in 75% of patients in one study of 8 patients with classic TN.

Levetiracetam has been tried in TN. The exact mechanism by which it acts is unknown, but it is thought to target high-voltage, N-type calcium channels as well as the synaptic vesicle protein 2A (SV2A); by this, it impedes impulse conduction across synapses. Its evidence in TN is scant. Recently, 2 pilot, open-label studies investigated the efficacy and tolerability of levetiracetam in patients with TN. One study with 10 patients reported an improvement of 50-90%. Another study with 23 patients reported a 62% reduction in number of daily attacks in patients receiving levetiracetam as add-on therapy.

Other drugs which have shown limited benefit in treating TN are also disclosed. For example, phenytoin and intravenous phenytoin is an anticonvulsant drug used in the prophylaxis and control of various types of seizures.

Fosphenytoin is a water-soluble phenytoin prodrug used only in hospitals for the treatment of epileptic seizures. It works by slowing down impulses in the brain that cause seizures. Its main mechanism is to block frequency-dependent, use-dependent and voltage-dependent neuronal sodium channels, and therefore limit repetitive firing of action potentials. Fosphenytoin may be used in the treatment of conditions including but not limited to trigeminal neuralgia.

Clonazepam is a long-acting benzodiazepine with intermediate onset commonly used to treat panic disorders, severe anxiety, and seizures. Clonazepam may be used in the treatment of conditions including but not limited to trigeminal neuralgia.

Valproic acid is an anticonvulsant used to control complex partial seizures and both simple and complex absence seizures. Valproic acid may be used in the treatment of conditions including but not limited to trigeminal neuralgia.

Misoprostol is a prostaglandin E1 analogue used to reduce the risk of NSAID-induced gastric ulcers and to terminate pregnancies. Misoprostol may be used in the treatment of conditions including but not limited to trigeminal neuralgia.

Tocainide is an orally active class 1b antiarrhythmic agent that interferes with cardiac sodium channels and typically used to treat ventricular arrhythmias. Tocainide may be used in the treatment of conditions including but not limited to trigeminal neuralgia.

Topical capsaicin cream is a topical analgesic agent used for the symptomatic relief of neuropathic pain associated with post-herpetic neuralgia, as well as other muscle and joint pain.

Intranasal lidocaine is a local anesthetic used in a wide variety of superficial and invasive procedures. It ultimately elicits its numbing activity by blocking sodium channels so that the neurons of local tissues that have the medication applied on are transiently incapable of signaling the brain regarding sensations. Intranasal lidocaine may be used in the treatment of conditions including but not limited to trigeminal neuralgia.

Tizanidine is an alpha-2 adrenergic agonist used for the short-term treatment of muscle spasticity. Tizanidine may be used in the treatment of conditions including but not limited to trigeminal neuralgia.

Sumatriptan is a serotonin receptor agonist used to treat migraines and cluster headaches. Sumatriptan may be used in the treatment of conditions including but not limited to trigeminal neuralgia.

Amitriptyline is a tricyclic antidepressant (TCA) with analgesic properties, widely used to treat depression and neuropathic pain. Amitriptyline, nortriptyline, and desipramine are commonly used off-label to treat PHN and CIPN. They provide analgesia by inhibiting the reuptake of serotonin and norepinephrine at the presynaptic nerve terminals thereby decreasing the sensory perception between the brainstem and spinal cord. TCAs should be started at a low dose of 10-25 mg at bedtime and gradually titrated every 3-7 days by 10-25 mg as tolerated by the patient up to a maximum dose of 150 mg daily. TCAs are associated with adverse effects like sedation, dry mouth, blurred vision, constipation, urinary retention, QT interval prolongation, sexual dysfunction, Xerostomia, water retention, increased appetite, weight gain, constipation, vertigo, and postural hypotension. Caution should be taken while prescribing TCAs to patients who are at risk of suicide and overdose. Amitriptyline is the most widely prescribed but has higher anticholinergic effects. Nortriptyline and desipramine are better tolerated and more effective than amitriptyline. The mechanism of action of TCAs is not fully elucidated. It is suggested that amitriptyline inhibits the membrane pump mechanism responsible for the re-uptake of transmitter amines, such as norepinephrine and serotonin, thereby increasing their concentration at the synaptic clefts of the brain.

Other pain conditions which may be treated by the compositions and methods described herein include complex regional pain syndrome and phantom limb pain. Complex regional pain syndrome may be treated by NSAIDs, anticonvulsants, tricyclic antidepressants, opioids, nerve stimulation, psychological support, and combinations thereof; such treatments may be replaced or used in combination with the compositions and methods of the present disclosure. Phantom limb pain may be treated by tricyclic antidepressants, serotonin-norepinephrine reuptake inhibitors, opioids and opioid-like medications, anticonvulsants, NMDA receptor agonists, local anesthetics, and combinations thereof; such treatments may be replaced or used in combination with the compositions and methods of the present disclosure.

Various formulations of an active agent for treatment of trigeminal neuralgia in the methods of the present disclosure are contemplated. For example, oral, sublingual, topical, and injectable formulations may be employed in the methods of the present disclosure.

Liquid formulations are contemplated. A typical liquid formulation may contain 6 mg/ml of the active substance, though other doses to reflect the dosing of the tablet form e.g. 2.5 mg/ml or 5 mg/ml could be used. Exact dosing may be established in Phase III studies of the specific indication if required. Other concentrations of the active substance could be used to ease administration or reduce the volume of non-active substances. The other non-active ingredients could be those typically used for syrup formulations sucrose, apricot flavor (no. 1NS), ethanol 96%, citric acid anhydrous, sodium citrate, sodium benzoate, sodium sulphite anhydrous (E221), sodium metabisulphite (E223), ascorbic acid and purified water. Substitutions and changes in the formulation, e.g. to reduce or eliminate sucrose could be investigated. For example, a liquid formulation may include 1 mg/mL of Trimeprazine tartrate in methanol.

Nasal spray formulations are also contemplated. The intranasal route is a very useful route of administration when CNS/Brain regions are the target of treatment. Medications for nasal decongestion, rhinitis, and migraine have been successfully delivered by the intranasal route. First generation antihistamines such as Trimeprazine or a pharmaceutically acceptable salt thereof cross the blood-brain-barrier (BBB), unlike second generation antihistamines, and they are not substrates for the P-glycoprotein efflux pump located in the cerebral endothelial cells of the blood-brain barrier.

Other first-generation antihistamines include ethylenediamines (such as mepyramine, chloropyramine, antazoline, and tripelennamine), which were the first group of clinically effective H₁-antihistamines developed.

Mepyramine, or pyrilamine, targets the H1 receptor. It is a first generation antihistamine. However, it rapidly permeates the brain and so often causes drowsiness as a side effect. It has been found in over-the-counter combination products for colds and menstrual symptoms but is considered to be an unapproved prescription medication used for cough, cold, or allergic conditions.

Chlorpyramine is a first-generation antihistamine used in Eastern European countries to treat bronchial asthma as well as allergic rhinitis, allergic conjunctivitis, and other allergic reactions. It is also indicated for Quincke's edema, allergic reactions to insect bites, food and drug allergies, and anaphylactic shock.

Antazoline is a 1st generation antihistamine with anticholinergic activity. It is used to relieve nasal congestion. It is also formulated as eye drops with naphazoline to relieve allergic conjunctivitis.

Tripelennamine is a histamine H1 antagonist with low sedative action but frequent gastrointestinal irritation. It is used to treat asthma; hay fever; urticaria; and rhinitis; and also in veterinary applications. Tripelennamine is administered by various routes, including topically.

Other first-generation antihistamines include ethanolamines (such as diphenhydramine, carbinoxamine, doxylamine, orphenadrine, bromazine, clemastine, and dimenhydrinate).

Diphenhydramine was the prototypical agent in this group. Significant anticholinergic adverse effects, as well as sedation, are observed in this group but the incidence of gastrointestinal adverse effects is relatively low. Diphenhydramine (perhaps known most commonly as its brand name formulation Benadryl) is a first-generation H1 receptor antihistamine that is used extensively for the treatment of seasonal allergies, insect bites and stings, and rashes. However, it also has antiemetic, antitussive, hypnotic, and anti-parkinsons properties. As histamine receptors exist both peripherally and in the central nervous system, diphenhydramine has been shown to cause sedation due to its competitive antagonism of histamine H1 receptors within the central nervous system. While its use in allergy therapy can sometimes fall out of favor due to its sedative effect, diphenhydramine has been repurposed for use within many non-prescription over-the-counter sleep aids and cough-and-cold medications that have been marketed for “nighttime” use. Diphenhydramine is also used in combination with 8-chlorotheophylline as the anti-nausea drug Dimenhydrinate where it is utilized primarily for its antagonism of H1 histamine receptors within the vestibular system. Diphenhydramine has also been shown to be implicated in a number of neurotransmitter systems that affect behavior including dopamine, norepinephrine, serotonin, acetylcholine, and opioid. As a result, diphenhydramine is being investigated for its anxiolytic and anti-depressant properties.

Carbinoxamine is a first-generation antihistamine that competes with free histamine for binding at HA-receptor sites. This antagonizes the effects of histamine on HA-receptors, leading to a reduction of the negative symptoms brought on by histamine HA-receptor binding. The product label for carbinoxamine as an over the counter cough and cold medicine is being modified to state “do not use” in children under 4 years of age in order to prevent and reduce misuse, as many unapproved carbinoxamine-containing preparations contained inappropriate labeling, which promoted unapproved uses (including management of congestion, cough, the common cold, and the use in children under 2 years of age), which can potentially cause serious health risks.

Doxylamine is a histamine H1 antagonist with pronounced sedative properties. It is used in allergies and as an antitussive, antiemetic, and hypnotic. Doxylamine has also been administered in veterinary applications and was formerly used in parkinsonism.

Orphenadrine is a muscarinic antagonist used to treat drug-induced parkinsonism and to relieve pain from muscle spasm. Orphenadrine can antagonize H1 receptors, N-methyl-D-aspartate receptor (NMDA) receptors, and non-selectively antagonize muscarinic acetylcholine receptors (hence its use as an anticholinergic). It also blocks the human Ether-à-go-go-Related Gene (HERG) potassium channel along with Nav1.7, Nav1.8, and Nav1.9 sodium channels, and is a norepinephrine and dopamine reuptake inhibitor.

Bromazine (also called Bromodiphenhydramine) is an ethanolamine antihistamine with antimicrobial property. Bromodiphenhydramine is used in the control of cutaneous allergies. Ethanolamine antihistamines produce marked sedation in most patients. Bromodiphenhydramine competes with free histamine for binding at HA-receptor sites. This antagonizes the effects of histamine on HA-receptors, leading to a reduction of the negative symptoms brought on by histamine HA-receptor binding.

Clemastine is an ethanolamine-derivative, first generation histamine H1 antagonist used in hay fever, rhinitis, allergic skin conditions, and pruritus. It causes drowsiness. Clemastine is a selective histamine H1 antagonist and binds to the histamine H1 receptor. This blocks the action of endogenous histamine, which subsequently leads to temporary relief of the negative symptoms brought on by histamine.

Dimenhydrinate is a medication used to prevent and treat nausea, vomiting, vertigo, and motion sickness. Early research into dimenhydrinate focused on its role as an antihistamine for urticaria; the treatment of motion sickness was an accidental discovery. Dimenhydrinate is a theoclate salt that separates into diphenhydramine and 8-chlorotheophylline. While the exact mechanism of action is unknown, diphenhydramine is theorized to reduce disturbances to equilibrium through antimuscarinic effects or histamine H1 antagonism. 8-chlorotheophylline may produce excitation through blocking adenosine receptors, reducing the drowsiness produced by diphenhydramine.

Other first-generation antihistamines include alkylamines (such as pheniramine, chlorpheniramine, dexchlorpheniramine, dexbrompheniramine, brompheniramine, triprolidine, dimetindene, and acrivastine). The isomerism is a significant factor in the activity of the agents in this group. E-triprolidine, for example, is 1000-fold more potent than Z-triprolidine. This difference relates to the positioning and fit of the molecules in the histamine H₁-receptor binding site. Alkylamines are considered to have relatively fewer sedative and gastrointestinal adverse effects, but relatively greater incidence of paradoxical central nervous system (CNS) stimulation.

Pheniramine is a first-generation antihistamine in the alkylamine class, similar to brompheniramine and chlorpheniramine. It is used in some over-the-counter allergy as well as cold & flu products in combination with other drugs. Pheniramine's use as an anti-allergy medication has largely been supplanted by second-generation antihistamines such as cetirizine and loratadine. Pheniramine competes with histamine for the histamine H1 receptor, acting as an inverse agonist once bound. The reduction in H1 receptor activity is responsible for reduced itching as well as reduced vasodilation and capillary leakage leading to less redness and edema. This can be seen in the suppression of the histamine-induced wheal (swelling) and flare (vasodilation) response. Inverse agonism of the H1 receptor in the CNS is also responsible for the sedation produced by first-generation antihistamines like pheniramine. The binding of pheniramine to H4 receptors, and subsequent inverse agonism, may also contribute to reduced itching by antagonizing inflammation.

Chlorpheniramine is a histamine H1 antagonist used in allergic reactions, hay fever, rhinitis, urticaria, and asthma. It has also been used in veterinary applications. One of the most widely used of the classical antihistamines, it generally causes less drowsiness and sedation than promethazine. Chlorpheniramine binds to the histamine H1 receptor. This blocks the action of endogenous histamine, which subsequently leads to temporary relief of the negative symptoms brought on by histamine.

Dexchlorpheniramine is a chlorpheniramine enantiomer antihistamine indicated in the treatment of sunburns, insect bites, and allergic reactions of the skin. Dexchlorpheniramine is a potent S-enantiomer of chlorpheniramine. The salt form dexchlorpheniramine maleate as the active ingredient is available as a prescription drug indicated for adjunctive therapy for allergic and anaphylactic reactions. It is an antihistamine drug with anticholinergic (drying) and sedative actions. It disrupts histamine signaling by competing with histamine for cell receptor sites on effector cells.

Dexbrompheniramine maleate is an antihistamine agent that is used for the treatment of allergic conditions, such as hay fever or urticaria. Dexbrompheniramine competitively binds to the histamine H1-receptor. It competes with histamine for the normal H₁-receptor sites on effector cells of the gastrointestinal tract, blood vessels and respiratory tract. This blocks the action of endogenous histamine, which subsequently leads to temporary relief of the negative symptoms brought on by histamine.

Brompheniramine is a histamine H1 antagonist used to treat coughs, upper respiratory symptoms, and nasal congestion associated with allergies and the common cold. Brompheniramine is an antagonist of the H1 histamine receptors with moderate antimuscarinic actions, as with other common antihistamines such as diphenhydramine. Due to its anticholinergic effects, brompheniramine may cause drowsiness, sedation, dry mouth, dry throat, blurred vision, and increased heart rate.

Triprolidine is a first-generation histamine H1 antagonist used in allergic rhinitis; asthma; and urticaria. It is a component of cough and cold medicines. It may cause drowsiness. riprolidine, is a histamine H1 antagonist that competes with histamine for the normal H1-receptor sites on effector cells of the gastrointestinal tract, blood vessels and respiratory tract. It provides effective, temporary relief of sneezing, watery and itchy eyes, and runny nose due to hay fever and other upper respiratory allergies. Triprolidine has anticholinergic and sedative effects.

Dimetindene is an antihistamine/anticholinergic used orally and locally as an antipruritic. Dimethindene occurs as a racemic mixture. The(S)-(+)-dimethindene is a potent M2-selective muscarinic receptor antagonist (with lower affinity for M1, M3, and M4 muscarinic receptors). The (R)-(−)-enantiomer is the eutomer (responsible for bioactivity) for histamine H1 receptor binding. Dimethindene is a selective histamine H1 antagonist and binds to the histamine H1 receptor. This blocks the action of endogenous histamine, which subsequently leads to temporary relief of the negative symptoms brought on by histamine.

Acrivastine is a triprolidine analog antihistamine indicated for the treatment of allergies and hay fever. As an H1 receptor antagonist, it functions by blocking the action of histamine at this receptor thereby preventing the symptoms associated with histamine release such as pruritis, vasodilation, hypotension, edema, bronchoconstriction, and tachycardia.

Other first-generation antihistamines include piperazines (such as cyclizine, buclizine, chlorcyclizine, hydroxyzine, meclizine, and ketotifen). These compounds are structurally related to the ethylenediamines and the ethanolamines, and produce significant anticholinergic adverse effects with the exception of hydroxyzine, which has low to no affinity for muscarinic acetylcholine receptors and therefore produces negligible anticholinergic side-effects. Compounds from this group are often used for motion sickness, vertigo, nausea, and vomiting. The second-generation H1-antihistamine cetirizine also belongs to this chemical group.

Cyclizine is a histamine H1 antagonist given by mouth or parenterally for the control of postoperative and drug-induced vomiting and in motion sickness. Cyclizine is a piperazine-derivative antihistamine used as an antivertigo/antiemetic agent. Cyclizine is used in the prevention and treatment of nausea, vomiting, and dizziness associated with motion sickness. Additionally, it has been used in the management of vertigo in diseases affecting the vestibular apparatus. Although the mechanism by which cyclizine exerts its antiemetic and antivertigo effects has not been fully elucidated, its central anticholinergic properties are partially responsible. The drug depresses labyrinth excitability and vestibular stimulation, and it may affect the medullary chemoreceptor trigger zone. It also possesses anticholinergic, antihistaminic, central nervous system depressant, and local anesthetic effects. Cyclizine acts to block the histamine receptors in the vomiting center and thus reduce activity along these pathways. Furthermore, since cyclizine possesses anti-cholinergic properties as well, the muscarinic receptors are similarly blocked.

Buclizine is an antihistamine medication with both antiemetic and anticholinergic effects. It was touted to be effective as an appetite stimulant in children when administered in the syrup form, however, this indication has not been validated. In addition to the above conditions, buclizine has been studied in the treatment of migraine attacks and in the treatment of nausea and vomiting during pregnancy. Buclizine acts to block the histamine receptors in the vomiting center and thus reduce activity along these pathways. Furthermore, since buclizine possesses anti-cholinergic properties as well, the muscarinic receptors are similarly blocked.

Chlorcyclizine is a first generation phenylpiperazine class antihistamine used to treat urticaria, rhinitis, pruritus, and other allergy symptoms. Chlorcyclizine also has some local anesthetic, anticholinergic, and antiserotonergic properties, and can be used as an antiemetic.

Hydroxyzine is a first-generation histamine H1-receptor antagonist of the dephenylmethane and piperazine classes that exhibits sedative, anxiolytic, and antiemetic properties. Hydroxyzine blocks the activity of histamine to relieve allergic symptoms such as pruritus. Activity at off-targets also allows for its use as a sedative anxiolytic and an antiemetic in certain disease states. Hydroxyzine is a potent inverse agonist of histamine H1-receptors-inverse agonists are agents that are considered to have a “negative efficacy”, so rather than simply blocking activity at a receptor they actively dampen its activity. Inverse agonism at these receptors is responsible for hydroxyzine's efficacy in the treatment of histaminic edema, flare, and pruritus. Hydroxyzine is not a cortical depressant, so its sedative properties likely occur at the subcortical level of the CNS. These sedative properties allow activity as an anxiolytic. Antiemetic efficacy is likely secondary to activity at off-targets. It is also a Potassium voltage-gated channel subfamily H member 2 inhibitor.

Meclizine is a histamine H1 antagonist with antiemetic and antivertigo properties. It is used in the symptomatic treatment of motion sickness and control of vertigo associated with vestibular system diseases. It also exhibits anticholinergic, central nervous system depressant, and local anesthetic effects. Through its antagonistic action on the H1 receptors, meclizine primarily works by inhibiting signaling pathway transduction through histaminergic neurotransmission from the vestibular nuclei and nucleus of the solitary tract (NTS) to the chemoreceptor trigger zone (CTZ) and medullary vomiting center. In addition to the Histamine H1 receptor, it is also an inverse agonist of Nuclear receptor subfamily 1 group I member 3.

Ketotifen is a benzocycloheptathiophene derivative with potent antihistaminic and mast cell stabilizing properties. It has a similar structure to some other first-generation antihistamines such as cyproheptadine and azatadine. The precise mechanism(s) through which ketotifen exerts its therapeutic effects are unclear. Ketotifen is a potent and non-competitive antagonist of H1 histamine receptors, which is likely to be a significant contributor to its anti-allergic activity. In addition, ketotifen stabilizes mast cells and has demonstrated in vitro the ability to inhibit the release of allergic and inflammatory mediators such as histamine, leukotrienes C₄ and D₄ (i.e. SRS-A), and platelet-activating factor (PAF).

Other first-generation antihistamines include tricyclics and tetracyclics (such as promethazine, alimemazine, cyproheptadine, and mequitazine). These compounds differ from the phenothiazine antipsychotics in the ring-substitution and chain characteristics. They are also structurally related to the tricyclic antidepressants (and tetracyclics), explaining the H₁-antihistaminergic adverse effects of those three drug classes and also the poor tolerability profile of tricyclic H₁-antihistamines. The second-generation H₁-antihistamine loratadine was derived from compounds in this group.

Promethazine is a first-generation antihistamine. Promethazine antagonizes a variety of receptors, allowing it to be used for a number of indications including allergic reactions, pain, sedation, nausea, and vomiting. Promethazine is an antagonist of histamine H1, post-synaptic mesolimbic dopamine, alpha adrenergic, muscarinic, and N-methyl-D-aspartate receptor (NMDA) receptors. The antihistamine action is used to treat allergic reactions. Antagonism of muscarinic and NMDA receptors contribute to its use as a sleep aid, as well as for anxiety and tension. Antagonism of histamine H1, muscarinic, and dopamine receptors in the medullary vomiting center make promethazine useful in the treatment of nausea and vomiting.

Alimemazine, also known as Trimeprazine is a first generation antihistamine used to prevent and relieve allergic conditions which cause pruritus and other allergic skin conditions, including urticaria. Trimeprazine competes with free histamine for binding at HA-receptor sites. This antagonizes the effects of histamine on HA-receptors, leading to a reduction of the negative symptoms brought on by histamine HA-receptor binding.

Cyproheptadine is a potent competitive antagonist of both serotonin and histamine receptors. It is used primarily to treat allergic symptoms, though it is perhaps more notable for its use in appetite stimulation and its off-label use in the treatment of serotonin syndrome. Cyproheptadine appears to exert its antihistamine and antiserotonin effects by competing with free histamine and serotonin for binding at their respective receptors. Antagonism of serotonin on the appetite center of the hypothalamus may account for cyproheptadine's ability to stimulate the appetite. In addition to the Histamine H1 receptor, Cyproheptadine is also an antagonist of 5-hydroxytryptamine receptor 2A, 5-hydroxytryptamine receptor 2C, Histamine H2 receptor, 5-hydroxytryptamine receptor 2B, Muscarinic acetylcholine receptor M1, Muscarinic acetylcholine receptor M2, Muscarinic acetylcholine receptor M3, and 5-hydroxytryptamine receptor 7.

Mequitazine is a histamine H1 antagonist (antihistamine). It competes with histamine for the normal H1-receptor sites on effector cells of the gastrointestinal tract, blood vessels and respiratory tract. It provides effective, temporary relief of sneezing, watery and itchy eyes, and runny nose due to hay fever and other upper respiratory allergies.

Formulation of compounds such as Trimeprazine into a nasal spray could deliver drug more directly to the brain and enable more efficient targeting of brain regions (dorsal root ganglion and gasserian ganglion) involved in the perception of neuropathic pain in the targeted indications. This would also have the potential advantage of reducing the dose needed for a therapeutic response vs. the CNS side effects of compounds such as Trimeprazine such as drowsiness. Compounds such as Trimeprazine would be amenable to formulation into a nasal spray. In some embodiments, the LogP of Trimeprazine is 4.71 making the compound lipophilic and according to Lipinski's rule of 5, compounds with LogP>5 are able to cross the BBB, without wishing to be bound by theory. A formulation of compounds such as Trimeprazine in a similar vehicle to Azelastine (benzalkonium chloride, edetate disodium, hypromellose, citric acid, dibasic sodium phosphate, sodium chloride, and purified water) could be used.

Sublingual dissolving film formulations are also contemplated. “Oral thin film,” “OTF,” “oral dissolving film,” “oral dissolvable film,” “oral drugstrip” or “oral strip” refers to a product used to administer active ingredients via absorption in the mouth (buccally or sublingually), the stomach(gastrically), and/or via the small intestines (enterically). The oral thin film is edible and pharmaceutically acceptable. A film is prepared typically using hydrophilic polymers that rapidly dissolve on the tongue or buccal cavity delivering the active ingredient to the systemic circulation via dissolution when contact with liquid is made. The oral thin film can also be used to adhere to mucosal tissue (e.g., at least one of mouth, nose, eye, vagina, and rectum), thereby locally delivering the active ingredient. As such, it is appreciated that those of skill in the art understand that reference to an oral thin film for use with mucosal tissue, such as nose, eye, vagina, and rectum is appropriate and acceptable. When put on the tongue, such films disintegrate instantaneously, releasing the drug which dissolves in the saliva. Some drugs are absorbed from the mouth, pharynx, and esophagus as the saliva passes down into the stomach. In such cases, the bioavailability of the drug is significantly greater than that observed for conventional tablets. For the treatment of TN, an oral thin film formulation of compounds such as Trimeprazine or a pharmaceutically acceptable salt thereof would be possible and could have useful advantages such as ease of administration/convenience especially for elderly patients, no need for water, potential for rapid absorption, potential for lower dose to achieve therapeutic efficacy vs. side effects. For the paroxysms of pain characteristic of TN, an oral thin film formulation of compounds such as Trimeprazine or a pharmaceutically acceptable salt thereof may be useful. In some embodiments, sublingual dissolving film formulations include oral thin films. In some embodiments, when put on the tongue, such films disintegrate instantaneously, releasing the drug which dissolves in the saliva, and is then absorbed via the oral mucosa directly into systemic circulation. In such cases, oral thin films can circumvent the first-pass metabolism effect, which increases the bioavailability of active ingredients, in some cases the bioavailability of the drug is significantly greater (50%) than that observed for conventional tablet formulations. Some drugs are absorbed from the mouth, pharynx, and esophagus as the saliva passes down into the stomach and later absorbed through the gastrointestinal system like traditional oral dosage forms. For the treatment of TN, and oral thin film would be particularly useful given the drug delivery characteristics of oral thin films. In some embodiments, an oral thin film may comprise about 1 mg to about 5 mg of Trimeprazine or a pharmaceutically acceptable salt thereof in one or more layers making up an oral thin film formulation.

In some embodiments, the use of an oral thin film comprising Trimeprazine or a pharmaceutically acceptable salt thereof for the treatment of TN may result in a local anesthetic effect (e.g., a numbing sensation in the oral cavity close to the site of two of the trigeminal nerve branches (V2, V3) which may have additional therapeutic benefits.

In some embodiments, the use of an oral thin film comprising Trimeprazine or a pharmaceutically acceptable salt thereof for the treatment of TN provides for ease of administration for TN patients undergoing a TN pain attack where the ability to use a traditional oral dosage form is difficult as the TN patient may be unable to swallow, open the mouth wide, drink, and may have a fear of choking. In some embodiments, the use of an oral thin film comprising Trimeprazine or a pharmaceutically acceptable salt thereof for the treatment of TN may be particularly convenient elderly patients with TN who may have limited ability to swallow a traditional oral dosage form because of TN, plus other comorbidities such as Alzheimer's that affect the swallowing reflex.

In some embodiments, the use of an oral thin film comprising Trimeprazine or a pharmaceutically acceptable salt thereof or a pharmaceutically acceptable salt thereof for the treatment of TN does not require water at the time of administration.

In some embodiments, the use of an oral thin film comprising Trimeprazine or a pharmaceutically acceptable salt thereof for the treatment of TN has the potential for rapid absorption into the systemic circulation and hence rapid onset of pain relief.

In some embodiments, the use of an oral thin film comprising Trimeprazine or a pharmaceutically acceptable salt thereof for the treatment of TN allows for the use of lower doses of Trimeprazine or a pharmaceutically acceptable salt thereof while still maintaining therapeutic efficacy in the treatment of pain from TN. By way of a non-limiting example, a lower dose (i.e., below the dose level used in the tablet form of Trimeprazine or a pharmaceutically acceptable salt thereof) could reduce the level of drowsiness while maintaining the same therapeutic effect. For example, a typical dose of Trimeprazine or a pharmaceutically acceptable salt thereof (5 mg BID) can cause drowsiness, In some embodiments, the use of an oral thin film comprising Trimeprazine or a pharmaceutically acceptable salt thereof for the treatment of TN comprising about 0.5 mg-2.5 mg Trimeprazine or a pharmaceutically acceptable salt thereof twice per day may avoid drowsiness altogether. In some embodiments, a lower dose could reduce the level of dizziness in elderly patients for the same therapeutic effect in treating the pain of TN.

In some embodiments, an oral thin film comprising Trimeprazine or a pharmaceutically acceptable salt thereof for the treatment of TN will be bioequivalent to a traditional oral tablet formulation of Trimeprazine or a pharmaceutically acceptable salt thereof.

In some embodiments, an oral thin film comprising Trimeprazine or a pharmaceutically acceptable salt thereof for the treatment of TN may comprise between about 0.5 mg to about 2.5 mg of Trimeprazine or a pharmaceutically acceptable salt thereof. In some embodiments, an oral thin film comprising Trimeprazine or a pharmaceutically acceptable salt thereof for the treatment of TN may comprise between about 0.5 mg to about 2.5 mg of Trimeprazine or a pharmaceutically acceptable salt thereof per film. In some embodiments, an oral thin film comprising Trimeprazine or a pharmaceutically acceptable salt thereof for the treatment of TN will have greater bioavailability in the brain. When administered using an oral thin film comprising Trimeprazine or a pharmaceutically acceptable salt thereof for the treatment of TN, Trimeprazine or a pharmaceutically acceptable salt thereof is rapidly absorbed via the transmucosal route, enters the systemic circulation and exerts a therapeutic affect on the pain of TN via the H1 histamine receptors in the brain (principally the tuberomamillary nucleus). This can result in therapeutic efficacy while also lacking some of the common side effects of Trimeprazine or a pharmaceutically acceptable salt thereof such as, but not limited to drowsiness.

In some embodiments the properties of an oral thin film comprising Trimeprazine or a pharmaceutically acceptable salt thereof for the treatment of TN is designed to provide a less rapidly absorbed therapeutically effective dose via the dissolution of the inner layer allowing the drug to be absorbed from the mouth, pharynx, and esophagus as the saliva passes down into the stomach and later absorbed through the GI system like traditional oral dosage forms.

In some embodiments, the oral thin film comprising Trimeprazine or a pharmaceutically acceptable salt thereof for the treatment of TN is a multi-layer oral thin film designed to provide a more rapid onset initial therapeutic effect via the transmucosal route, followed by a second “dose” and therapeutic effect via the traditional gastric route. Overall, this may reduce the total dose required for therapeutic effect vs. side effects, with the advantage for the patient of taking fewer oral thin films.

In some embodiments, the oral thin film comprising Trimeprazine or a pharmaceutically acceptable salt thereof for the treatment of TN may further comprise one or more polymers (including but not limited to water soluble polymers), plasticizers, surfactants, sweetening agents, saliva stimulating agents, super disintegrants, coloring agents, flavoring agents, stabilizing and thickening agents refreshing agents, antioxidants, mucoadhesives, plasticizers, or any combination thereof. In some embodiments, the polymer is selected from gum polysaccharides (e.g., gum arabic, sodium alginate), gelatin, maltodextrin, water-soluble cellulose derivatives such as but not limited to hydroxypropylmethylcellulose (HPMC) hydroxyethyl cellulose, hydroxypropyl cellulose, polyvinylalcohol (PVA), polyethylene oxide (PEO), polyvinyl pyrrolidone (PVP), chitosan, kollicoat, sodium alginate, pectin, rosin, water-soluble cellulose derivatives, copovidone (a copolymer of 1-vinyl-2-pyrrolidone and vinyl acetate); other copolymers of vinyl pyrrolidone; other polymers or copolymers of substituted vinyl pyrrolidone; derivatives of polyvinyl pyrrolidone; polyethylene oxide, carboxymethyl cellulose; polyvinyl alcohol; natural gums, including xanthan, tragacanth, guar, acacia and arabic gums; and water-soluble polyacrylates. Examples of substituted vinyl pyrrolidones include but are not limited to N-vinyl-3-methyl-2-pyrrolidone, N-vinyl-4-methyl-2-pyrrolidone, N-vinyl-5-methyl-2-pyrrolidone, N-vinyl-5,5-dimethyl-2-pyrrolidone, N-vinyl-3,3,5-trimethyl-2-pyrrolidone and others. Examples of monomers that can be copolymerized with vinyl pyrrolidone or substituted vinyl pyrrolidones include vinyl aromatic monomers such as styrene, and acrylate or methacrylate monomers such as methyl methacrylate and 2-dimethylaminoethyl methacrylate, or any combination thereof. In some embodiments, the polymer may be an agent that enhances oral thin film dissolution in the mouth and minimize tensile strength. In some embodiments, the plasticizer is selected from polyethylene glycol, propylene glycol, glycerol, and polyols. citric acid, phthalic acid esters, glycerol, diethylene glycol, Bis(2-ethylhexyl) phthalate, ethylene glycol, tributyl citrate, tri ethyl citrate or any combination thereof. In some embodiments, the plasticizer is an agent that improves the stability and brittleness of the oral thin film. Specifically, the term “surfactant” is intended to mean an amphophilic compound that lowers the surface tension of a liquid, the interfacial tension between two liquids, or the interfacial tension between a liquid and a solid. Combinations of surfactants can be used. In some embodiments, the surfactant is selected from sodium lauryl sulfate, polysorbates, poloxamer 407, polyoxy-ethylene sorbitan fatty acid esters, an α-hydro-co-hydroxypoly (oxyethylene) poly (oxypropylene) poly(oxyethylene) block copolymer, a polyoxyethylene allyl ether, a polyoxyethylene, a castor oil derivative glycol, glycerol, erythritol, threitol, arabitol, xylitol, ribitol, mannitol, sorbitol, galactitol, fucitol, iditol, inositol, volemitol, isomalt, maltitol, lactitol, maltotritol, maltotetraitol or any combination thereof. In some embodiments, the surfactant is an agent that functions as a wetting or dispersing agent, enabling the oral thin film to melt rapidly and release the Trimeprazine or a pharmaceutically acceptable salt thereof. In some embodiments, the sweetening agent is selected from acesulfame potassium, aspartame, aspartan-acesulfame salt, cyclamate, erythritol, glycerol, glycyrrhizin, hydrogenated starch hydrolysate, isomalt, lactitol, maltitol, mannitol, neotame, polydextrose, saccharin, sorbitol, sucralose, tagatose, xylitol, dextrose, glucose, fructose, honey, xylose, ribose, caffeine, sucrose, maltose, stevioside, sodium or calcium saccharin salts, thaumatin I and II, aspartame, fructose, sorbitol, mannitol, or any combination thereof. In some embodiments, the sweetening agent is an agent capable of increasing the palatability of the oral thin film. In some embodiments, the saliva stimulating agent is selected from citric acid, malic acid, lactic acid, ascorbic acid, tartaric acid, or any combination thereof. In some embodiments, the saliva stimulating agent is an agent capable of speeding up the disintegration of the oral thin film through increased saliva production. In some embodiments, the super disintegrant is agent capable of causing the rapid disintegration of the oral thin film through a combined effect of swelling and water absorption. In some embodiments, the coloring agents are selected from FD&C licensed coloring agents (FD&C Yellow #6 and titanium dioxide are examples), EU colors, natural coloring agents, pigments, or any combination thereof. In some embodiments, the flavoring agent may be selected from isoamyl acetate (banana flavor), benzaldehyde (cherry flavor), cinnamaldehyde (cinnamon flavor), ethyl propionate (fruit flavor), methyl anthranilate (grape flavor), limonene (orange flavor), ethyl decadienoate (pear flavor), allyl hexanoate (pineapple flavor), ethyl meltol, ethylanillin (vanilla flavor), and methyl salicylate (wintergreen flavor), menthol, chloroform, certain salts, or any combination thereof. In some embodiments, the stabilizing and thickening agent may be selected from natural gums (such as but not limited to xanthan gum, locust bean gum, carrageenan), cellulosic derivatives or combinations thereof. In some embodiments, the stabilizing and thickening agent enhance the viscosity and strength of the oral thin film preparation solution's dispersion or suspension before casting. In some embodiments, the refreshing agent can be a chemical that trigger the cold sensitive receptors creating a cold sensation. In some embodiments, refreshing agents are selected from menthol, thymol, camphor eucalyptol or combinations thereof. In some embodiments, antioxidants are selected from sulfite salts such as sodium sulfite, sodium bisulfite, sodium metabisulfite and analogous salts of potassium and calcium or combinations thereof. In some embodiments, oral thin films can advantageously employ an antioxidant or oxygen scavenger to prevent or reduce oxidative degradation of the active ingredient prior to use. In some embodiments, mucoadhesives can be selected from sodium alginate, sodium carboxymethyl cellulose, guar gum, hydroxyethyl cellulose, hydroxypropyl cellulose, hydroxypropylmethyl cellulose, karya gum, methylcellulose, polyethylene oxide, retene, tragacanth, or combinations thereof. In some embodiments, mucoadhesives promote adhesion of oral thin film to the oral mucosa.

Traditional tablet formulations are also contemplated. About 2.5 mg to about 10 mg of active agent per tablet may be an exemplary amount. Such a tablet may be administered one, two, or three times a day, or other dosing regimens as appropriate. Other non-active ingredients may include microcrystalline cellulose, lactose, colloidal anhydrous silica, magnesium stearate, sodium starch glycollate, hypromellose, macrogol 200, indigotin E132 and titanium dioxide E171. For example, a tablet may include 5 mg of Trimeprazine or a pharmaceutically acceptable salt thereof (as Trimeprazine tartrate).

Modified release formulations including but not limited to sustained release, sustained action, prolonged action, controlled release, time-released, and long-acting dosage forms of Trimeprazine or a pharmaceutically acceptable salt thereof are contemplated. In some embodiments, modified release formulations maintain a consistent serum drug concentration (SDC) to balance efficacy with potential side effects or adverse events. In some embodiments, a modified release formulation of Trimeprazine or a pharmaceutically acceptable salt thereof for the treatment of TN may be used as maintenance therapy and optionally in combination with an oral thin film formulation which may be used to treat acute episodes of TN. In some embodiments, the formulations described herein are modified release formulations selected from an extended-release formulation, a sustained release formulation, a controlled release formulation, or a delayed release formulation.

In some embodiments, the modified release formulation described herein is an inert solid vehicle, or matrix, in which a drug is uniformly suspended, including in the form of tablets or small beads. In some embodiments, the matrix is a gelling material including gelatin, methylcellulose, gum tragacanth, Veegum, and alginic acid. In some embodiments, the matrix is a polymer including polylactic acid copolymer, polyacrylate, methacrylate, polyester, ethylene-vinyl acetate copolymer (EVA), polyglycolide, polylactide, and silicone. In some embodiments, the modified release formulation is a slow-release pellet, bead, or granule. In some embodiments, the modified release formulation is an extended-release tablet where the solubility of a drug is modified for extended release. In some embodiments, the extended-release tablet is formed by using the nonionized base or acid form of the drug. In some embodiments, the extended release tabled is formed by granulating the drug with excipients (including stearic acid, castor wax, high-molecular-weight polyethylene glycol (Carbowax), glyceryl monosterate, white wax, spermaceti oil, magnesium stearate and hydrogenated vegetable oil (Sterotex)) to decrease the aqueous solubility of the drug. In some embodiments, the modified release formulation is an ion-exchange preparation whereby an anionic or cationic drug is complexed with an oppositely charged ionic resin to form an insoluble nonabsorbable resin-drug complex.

In some embodiments modified release formulations described herein further comprise one or more pharmaceutically acceptable excipients. Examples of pharmaceutically acceptable excipients that may be present in the composition include but are not limited to fillers/vehicles, solvents/co-solvents, preservatives, antioxidants, suspending agents, surfactants, antifoaming agents, buffering agents, chelating agents, sweeteners, flavoring agents, binders, extenders, disintegrants, diluents, lubricants, fillers, wetting agents, glidants, and combinations thereof.

In some embodiments, the modified release formulations described herein can further comprise one or more exemplary fillers. Examples of exemplary fillers include cellulose and cellulose derivatives such as microcrystalline cellulose, powdered cellulose; dextrates; starches such as dry starch, hydrolyzed starch, and starch derivatives such as corn starch; cyclodextrin; sugars such as powdered sugar and sugar alcohols such as lactose, mannitol, sucrose and sorbitol; inorganic fillers such as aluminum hydroxide gel, calcium carbonate (granules or powder), precipitated calcium carbonate, carbonate, magnesium aluminometasilicate, dibasic calcium phosphate; and sodium chloride, silicon dioxide, silicic acid, titanium dioxide, titanium oxide, dicalcium phosphate dihydrate, calcium sulfate, alumina, kaolin, talc, or combinations thereof.

In some embodiments, the modified release formulations described herein further comprise one or more disintegrants. Examples of disintegrants include starches, alginic acid, crosslinked polymers such as crosslinked polyvinylpyrrolidone, croscarmellose sodium, potassium starch glycolate, sodium starch glycolate, clays, celluloses, starches, gums, or combinations thereof.

In some embodiments, the modified release formulations described herein further comprise one or more binders, including but not limited to celluloses such as hydroxypropylcellulose, methyl cellulose, and hydroxypropylmethylcellulose; starches such as corn starch, pregelatinized starch, and hydroxypropyl starch; waxes and natural and synthetic gums such as acacia, tragacanth, sodium alginate; synthetic polymers such as polymethacrylates and polyvinylpyrrolidone; and povidone, dextrin, pullulane, agar, gelatin, tragacanth, macrogol, or combinations thereof.

In some embodiments, the modified release formulations described herein further comprise one or more wetting agents, including but not limited to oleic acid, glyceryl monostearate, sorbitan mono-oleate, sorbitan monolaurate, triethanolamine oleate, polyoxyethylene sorbitan mono-oleate, polyoxyethylene sorbitan monolaurate, sodium oleate, sodium lauryl sulfate, poloxamers, poloxamer 188, polyoxyethylene ethers, polyoxyethylene sorbitan fatty acid esters, polyoxyethylene fatty acid esters, polyethylene glycol fatty acid esters, polyoxyethylene hardened castor oil, polyoxyethylene alkyl ethers, polysorbates, cetyl alcohol, glycerol fatty acid esters (for example, triacetin, glycerol monostearate, etc.), polyoxymethylene stearate, sodium lauryl sulfate, sorbitan fatty acid esters, sucrose fatty acid esters, benzalkonium chloride, polyethoxylated castor oil, and combinations thereof.

In some embodiments, the modified release formulations described herein further comprise one or more lubricants, including but not limited to stearic acid, magnesium stearate, calcium hydroxide, talc, corn starch, sodium stearyl fumarate, alkali-metal and alkaline earth metal salts, waxes, boric acid, sodium benzoate, sodium acetate, sodium chloride, leucine, polyethylene glycol (PEG), a methoxypolyethylene glycol, propylene glycol, sodium oleate, glyceryl behenate, glyceryl palmitostearate, glyceryl benzoate, magnesium lauryl sulfate, sodium lauryl sulfate, and combinations thereof.

In some embodiments, the modified release formulations described herein further comprise one or more glidants, including but not limited to silica (colloidal anhydrous), starch, talc, magnesium stearate, calcium stearate, zinc stearate, dibasic calcium phosphate, magnesium carbonate, magnesium oxide, calcium silicate, silicon dioxide, silicon dioxide, colloidal silicon dioxide, talc, sodium lauryl sulfate, native starch, or combinations thereof.

In some embodiments, the modified release formulations described herein, may be a tablet and further comprises a top coat, such as hydroxypropyl-methylcellulose coating or polyvinyl alcohol coating, and are available under the trade name Opadry, such as Opadry White, Opadry II (Opadry is a registered trademark of BPSI Holdings LLC, Wilmington, DE, USA).

In some embodiments, the modified release formulations described herein can further comprise one or more preservative agents. Examples of preservative agents include sodium benzoate, paraoxybenzoic acid esters, methyl, ethyl, butyl, and propyl parabens, chlorobutanol, benzyl alcohol, phenylethylalcohol, dehydroacetic acid, sorbic acid, benzalkonium chloride (BKC), benzethonium chloride, phenol, phenylmercuric nitrate, thimerosal, or combinations thereof. Preservative agents can be included in the liquid dosage form. The preservative agents can be in an amount sufficient to extend the shelf-life or storage stability, or both, of the liquid dosage form.

In some embodiments, the modified release formulations described herein can further comprise one or more flavoring agents. Examples of flavoring agents include synthetic flavor oils and flavoring aromatics and/or natural oils, extracts from plants leaves, flowers, fruits, and so forth and the like or any combinations thereof. Additional examples include cinnamon oil, oil of wintergreen, peppermint oils, clove oil, bay oil, anise oil, eucalyptus, thyme oil, cedar leaf oil, oil of nutmeg, oil of sage, oil of bitter almonds, and cassia oil and the like or any combinations thereof. Also useful as flavors are vanilla, citrus oil, including lemon, orange, grape, lime and grapefruit, and fruit essences, including apple, banana, pear, peach, strawberry, raspberry, cherry, plum, pineapple, apricot, strawberry flavor, tutti-fruity flavor, mint flavor, or any combinations thereof.

In some embodiments, the modified release formulation can generally be in any physical form suitable for use in treating a subject. These forms can be referred to as a unit dosage form, such as an individual pill or tablet. In some examples, the pharmaceutical compositions can be formulated as tablets, capsules, granules, powders, liquids, suspensions, gels, syrups, slurries, suppositories, patches, nasal sprays, aerosols, injectables, implantable sustained-release formulations, or mucoadherent films. In some examples, the pharmaceutical formulation may be formed as a tablet, a bi-layer tablet, a capsule, a multiparticulate, a drug coated sphere, a matrix tablet, or a multicore tablet. A physical form can be selected according to the desired method of treatment.

In some embodiments, the modified release formulations described herein can be manufactured by various conventional methods such as conventional mixing, dissolving, granulating, dragee-making, levigating, emulsifying, encapsulating, entrapping, or lyophilizing processes. Modified release formulations can be formulated in a conventional manner using one or more physiologically acceptable carriers, diluents, excipients or auxiliaries that facilitate processing of the active agent into preparations that can be used pharmaceutically. Proper formulation can be selected upon the oral route of administration chosen.

In some embodiments, the modified release formulations described herein are a core tablet, or a tablet within a tablet, whereby the inner core is used for the slow-drug-release component, and the outside shell contains a rapid-release dose of drug. In some embodiments, the modified release formulation is achieved via microencapsulation whereby microscopic drug particles are encapsulated with a special coating material, such as ethylcellulose. In some embodiments, the modified release formulation is an osmotic drug delivery system in the form of a tablet which contains an outside semipermeable membrane and an inner core filled with a mixture of drug and osmotic agent (salt solution). In some embodiments, the modified release formulation is a gastroretentive system that can remain in the gastric region for several hours and prolong the gastric residence time of a drug. In some embodiments, the modified release formulation is a combination of any of the above-described embodiments.

In some embodiments, the modified release formulations described herein are for oral administration, the modified release formulation can combine Trimeprazine or a pharmaceutically acceptable salt thereof or a pharmaceutically acceptable salt thereof with another pharmaceutical agent with one or more pharmaceutically acceptable carriers well known in the art. Such carriers facilitate formulation as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques.

In some embodiments, the modified release formulations described herein may comprise a matrix selected from microcrystalline cellulose, sodium carboxymethylcellulose, hydroxyalkylcelluloses such as hydroxy propyl methylcellulose and hydroxypropylcellulose, polyethylene oxide, alkylcelluloses such as methylcellulose and ethylcellulose, polyethylene glycol, polyvinylpyrrolidone, cellulose acetate, cellulose acetate butyrate, cellulose acetate phthalate, cellulose acetate trimellitate, polyvinyl acetate phthalate, polyalkylmethacrylates, polyvinyl acetate and mixtures thereof.

Topical formulations such as creams, lotions, liquid drops, and the like are also contemplated. Compounds such as Trimeprazine or a pharmaceutically acceptable salt thereof could be formulated into a lotion or cream for topical application to the skin for the treatment of the pain of post-herpetic neuralgia (PHN), trigeminal neuralgia (TN), diabetic neuropathic pain (DNP) and chemotherapy-induced neuropathic pain (CIPN) for example. Standard methods of formulating cream, lotions or liquids could be used as is common in antihistamine and capsaicin creams. Other topical delivery systems, such as patches, are further contemplated. Patches are commonly comprised of an adhesive material containing the active substance (in an aqueous or other form of base), which is applied to a non-woven polyester felt backing and covered with a polyethylene terephthalate (PET) film release liner. Patches commonly contain the following inactive ingredients (but are not limited to): dihydroxyaluminum aminoacetate, disodium edetate, gelatin, glycerin, kaolin, methylparaben, polyacrylic acid, polyvinyl alcohol, propylene glycol, propylparaben, sodium carboxymethylcellulose, sodium polyacrylate, D-sorbitol, tartaric acid, and urea. Compounds such as Trimeprazine or a pharmaceutically acceptable salt thereof could be formulated into a patch for topical application to the skin for the treatment of the pain of PHN, TN, DNP and CIPN for example. Compounds which are absorbed by the skin could also have a systemic effect as well as a local effect on pain.

Subcutaneous or intrathecal injection is also contemplated. Compounds such as Trimeprazine or a pharmaceutically acceptable salt thereof could be formulated for subcutaneous injection to treat the pain of PHN, DNP, TN and CIPN. Standard formulations and syringes/needles suitable for subcutaneous injection could be used. There is some evidence that intrathecal injection of drugs such as methylprednisolone and lidocaine can suppress the pain of PHN due to the effect of such drugs on the reduction in transmission of pain signals from the afferent nerves to the dorsal root ganglion (DRG). Compounds such as Trimeprazine or a pharmaceutically acceptable salt thereof could be formulated for the intrathecal route using common formulations and delivery technology (pumps, needles etc.). Pre-clinical and clinical formulation, efficacy and dosing studies would be needed in order to demonstrate therapeutic value (reduction in pain).

The inventors of the present disclosure determined that the off-label treatments effective in post-herpetic neuralgia (PHN) may be effective in treating trigeminal neuralgia (TN) because both are neuropathic pain conditions. Both conditions may be driven by latent infections, such as Herpes zoster (shingles), Borrelia burgdorferi (Lyme Disease). Post-herpetic pain involves the dorsal root ganglion. Trigeminal neuralgia involves the trigeminal ganglion (TG). Both the DRG and the TG are involved in the perception of itch and pain. The thalamus plays a key role in the transmission of pain and itch sensations from the DRG and especially TN pain via the TG. The DRG and TG share similar pathways and signaling molecules associated with pain and itch. In the context of Trimeprazine the DRG, TG, and thalamus express histamine receptors. Direct neuromodulation of the DRG has shown promising results in the treatment of PHN and painful diabetic peripheral neuropathy (DPN). Neuromodulation of the TG directly via a variety of approaches (e.g. chemical, electrical, surgical and pharmacological) is also a promising strategy for the treatment of TN as is neuromodulation of the thalamus. There is considerable overlap in the cellular signaling pathways involved in both itch and pain. For instance, transient receptor potential ion channels—subtypes V1 (TRPV1) and A1 (TRPA1)—as well as Toll-like receptors (TLRs) and proteinase-activated receptors (PARs) are implicated in both sensations.

Applicant has demonstrated efficacy in successfully treating patients (n=7) for TN with Trimeprazine and provided clinical evidence (non-randomized clinical trial) in support of the present disclosure.

Without wishing to be bound by theory, the following rationale is contemplated as a potential mechanism of action for Trimeprazine in Trigeminal Neuralgia.

### Pathological Mechanisms in Trigeminal Neuralgia

Trigeminal neuralgia (TN) arises from damage or inflammation of the trigeminal nerve or its branches, involving dysfunction in both the peripheral nervous system (PNS) and central nervous system (CNS). The pathophysiology of TN is complex and multifactorial, encompassing several molecular and cellular processes. A key mechanism involves inflammatory responses triggered by tissue damage surrounding the trigeminal nerve in the PNS, leading to peripheral sensitization and the hallmark symptoms of allodynia and hyperalgesia.

Peripheral sensitization is driven by inflammatory mediators, such as prostaglandin E2 (PGE2), cytokines, and neuropeptides like brain-derived neurotrophic factor (BDNF), released by immune cells—including macrophages, neutrophils, and mast cells. Among these, histamine released from mast cells plays a crucial role in sensitizing peripheral nerve endings by depolarizing nociceptors. Alterations in the expression of nociceptive receptors, such as transient receptor potential vanilloid 1 (TRPV1) and transient receptor potential cation channel subfamily A member 1 (TRPA1), result in increased excitability of trigeminal neurons. Additionally, changes in sodium, potassium, and calcium channel activity lead to the activation and proliferation of satellite glial cells (SGCs), further amplifying neuronal excitability. This increased excitability in trigeminal ganglion (TG) neurons enhances the release of neuropeptides such as substance P (SP) and calcitonin gene-related peptide (CGRP) into the brainstem.

Peripheral sensitization may also involve demyelination of the trigeminal nerve due to nerve compression which can result in ephaptic transmission and the associated symptoms of allodynia and hyperalgesia. The failure of the nerve to remyelinate may be exacerbated by an aberrant inflammatory response, potentially reinforcing the cycle of sensitization, particularly in patients with conditions such as multiple sclerosis.

The heightened sensitization of trigeminal neurons in the PNS triggers neuroplastic changes and hyperexcitability in central neurons, leading to central sensitization. Many of the molecular processes underlying central sensitization mirror those in the periphery. Persistent and elevated input from the central terminus of the trigeminal nerve increases the synaptic release of glutamate, SP, and CGRP within the trigeminal spinal nucleus caudalis, which also activates glial cells, particularly astrocytes. In parallel, a reduction in GABA and glycine signaling within local circuits, along with altered expression of potassium chloride cotransporter 2 (KCC2) and GABA transporters, diminishes inhibitory control and heightens neuronal excitability. Additionally, descending inhibitory modulation from somatosensory efferents is reduced, further contributing to central sensitization.

### Antihistamines

Trimeprazine tartrate, like many first-generation antihistamines, is a potent blocker of the histamine H1 (H1) receptor. Similar to other members of the G-protein-coupled receptor (GPCR) family, the histamine H1 receptor functions as a cellular switch, existing in equilibrium between the inactive (“off”) state and the active (“on”) state. Unlike histamine, first-generation antihistamines are not structurally related to histamine and do not compete for the same binding site. Instead, they bind to alternative sites on the receptor, producing the opposite effect. Therefore, H1-antihistamines are not simply receptor antagonists but function as inverse agonists, stabilizing the receptor in its inactive conformation and reducing the likelihood of excessive stimulation by histamine.

In addition to blocking histamine activity, H1-antihistamines inhibit the NF-κB transcription factor involved in the immune response through the phospholipase C and phosphatidylinositol (PIP2) signaling pathways. This leads to decreased antigen presentation and a reduction in the expression of pro-inflammatory cytokines, cell adhesion molecules, and chemotactic factors. Moreover, by lowering intracellular calcium concentrations, H1-antihistamines promote mast cell stability, thereby reducing further histamine release.

Due to their off-target effects, including antimuscarinic, anti-α-adrenergic, and anti-serotonergic activity, and their ability to cross the blood-brain barrier (BBB), first-generation antihistamines such as Trimeprazine have therapeutic applications beyond managing H1 histamine-mediated allergic reactions. These effects have enabled their use as antitussives, antiemetics, anti-nausea agents, sedatives, and treatments for Parkinson's disease.

### Role of Histamine and Histamine H1 Receptors (H1R) in Neuropathic Pain

Histamine plays a critical role in various physiological processes, including inflammatory responses, allergic reactions, and pain modulation. The histamine H1 receptor (H1R), a G-protein-coupled receptor (GPCR), is one of four histamine receptor subtypes (H1R, H2R, H3R, and H4R) and is primarily involved in mediating pro-inflammatory responses, including pain.

When histamine binds to H1R, it induces a conformational change in the receptor, leading to the activation of the associated Gq/11 protein. This activation triggers the dissociation of the G-protein into its subunits, Gαq/11 and Gβγ, which initiate downstream signaling cascades. The Gαq/11 subunit activates phospholipase C-β (PLC-β), an enzyme that hydrolyzes phosphatidylinositol 4,5-bisphosphate (PIP2) in the cell membrane into two secondary messengers: inositol 1,4,5-trisphosphate (IP3) and diacylglycerol (DAG). IP3 binds to its receptors on the endoplasmic reticulum (ER), leading to the release of calcium ions (Ca²⁺) into the cytosol, a key event in H1R-mediated signaling. The rise in intracellular Ca²⁺ activates calcium-dependent enzymes, including protein kinase C (PKC), and Ca²⁺ also forms a complex with calmodulin. Calmodulin, in turn, suppresses the activity of potassium voltage-gated channels type 7 (Kv7) and sensitizes transient receptor potential vanilloid 1 (TRPV1) receptors. DAG remains in the plasma membrane, where it works alongside Ca²⁺ to activate PKC, a pivotal player in regulating ion channels, gene expression, and the sensitization of nociceptive neurons.

PKC activation leads to the phosphorylation of various proteins, including ion channels and receptors involved in nociception, such as TRPV1, sodium channels, and potassium channels. This phosphorylation enhances the sensitivity of nociceptive neurons to painful stimuli, contributing to the perception of pain. This process is particularly relevant in neuropathic pain, where these pathways can become aberrantly activated. Activation of TRPV1, a non-selective cation channel, allows the influx of sodium, potassium, and calcium ions, resulting in neuronal depolarization and increased excitability, manifesting as heightened pain perception.

Inflammatory processes underlying neuropathic pain, such as those caused by tissue damage, insult, or infection (e.g., Herpes zoster), activate both resident immune cells, such as mast cells, and recruit immune cells, such as polymorphonuclear leukocytes (PMNLs), as well as other cell types, including epithelial cells, Schwann cells, fibroblasts, and sympathetic post-ganglionic neurons (SPGN). Mast cells, in particular, release histamine, which binds to H1R on peripheral afferents and in the dorsal root ganglia (DRG) and trigeminal ganglia (TG). This activates TRPV1, creating a feedback loop that perpetuates inflammation and pain. Moreover, H1R activation in peripheral nerves can lead to neurogenic inflammation, where the release of neuropeptides such as substance P further sensitizes pain pathways.

In the central nervous system (CNS), histamine acting on H1 receptors in the DRG, TG, and thalamus contributes to central sensitization, a hallmark of chronic neuropathic pain. Central sensitization involves the amplification of synaptic transmission within pain pathways, leading to exaggerated responses to typically non-painful (allodynia) or mildly painful stimuli (hyperalgesia)—a key feature of trigeminal neuralgia.

### Commonalities Between Itch and Pain

Itch and pain share significant commonalities despite being distinct sensations. Pain is known to suppress itch, while analgesics like morphine can paradoxically provoke itching. Both chronic pain and chronic itch exhibit similar pathological mechanisms, especially in prolonged conditions. These sensations are detected by primary sensory neurons located in the dorsal root ganglion (DRG) and the trigeminal ganglion. Pruriceptors (itch receptors) and nociceptors (pain receptors) overlap within the DRG, with pruriceptors representing a subset of the C-fiber nociceptors.

There is considerable overlap in the cellular signaling pathways involved in both itch and pain. For instance, transient receptor potential ion channels—subtypes V1 (TRPV1) and A1 (TRPA1)—as well as Toll-like receptors (TLRs) and proteinase-activated receptors (PARs) are implicated in both sensations. However, some receptors, such as the G-protein-coupled receptor (GPCR) MrgprA3 (MAS-related GPR, member A3) and the thymic stromal lymphopoietin (TSLP) receptor, are specific to itch. TRPV1 plays a crucial role in itch induced by histamine, the best known endogenous pruritogen. Histamine type 1 receptor (H1R) coexpresses with TRPV1 in pruritogenic nociceptors, and its signaling is coupled to TRPV1 activation, inducing membrane depolarization and activation of Ca2+-dependent intracellular cascades.

Conditions like neuropathies and dermatitis, which alter the sensitization of the peripheral nervous system (PNS) and central nervous system (CNS) (brainstem, spinal cord, and cortical neurons), can result in both pain and itch. For example, central sensitization underlies both touch-evoked pain (allodynia) and touch-evoked itch (alloknesis). Both sensations are also influenced by the immune system, as inflammatory mediators released in response to tissue damage or injury—such as insect bites (itch) or infections (e.g., Herpes zoster)—can directly activate or sensitize pruriceptive and nociceptive neurons, leading to heightened sensitivity to itch and pain. Oxidative stress has been strongly implicated in the pathogenesis of chronic pain and has also been shown to induce histamine-independent itch through the activation of TRPA1.

Sodium channels play a critical role in both itch and pain. Gain-of-function and loss-of-function mutations in the sodium channel subunit Nav1.7 have been strongly implicated in human pain sensation, along with Nav1.8 and Nav1.9. Notably, Nav1.7 has also been reported to be involved in paroxysmal itch.

Chronic pain is a well-recognized hypersensitivity state resulting from peripheral and central sensitization. Similarly, chronic itch and chronic cough are increasingly being understood as hypersensitivity syndromes. First-generation antihistamines, as previously discussed, have a broad range of therapeutic applications, particularly for itch due to their central and peripheral antihistaminic effects. Their off-target effects, including antimuscarinic, anti-α-adrenergic, and anti-serotonergic activity, have also made them useful in the management of chronic cough and pain. For example, Trimeprazine has been employed for pruritus, as a mild sedative, and anti-emetic.

### The Role of Transient Receptor Potential Vanilloid Type-1 (TRPV1) in Pain

The transient receptor potential vanilloid type-1 (TRPV1) cation channel is critical in the perception and modulation of pain. The TRPV1 receptor is activated by various stimuli, including noxious heat, deviations in pH (both acidic and basic), voltage changes, and endogenous compounds such as endocannabinoids and lipoxygenase products. Capsaicin and resiniferatoxin are two of the most well-known activators of TRPV1. Upon activation of TRPV1 in rodent and human trigeminal primary sensory neurons, pain-related neuropeptides—such as calcitonin gene-related peptide (CGRP) and substance P (SP)—are released from both peripheral and central nerve endings. These neuropeptides subsequently bind to their respective receptors on effector cells, leading to neurogenic inflammation and sensitization of nociceptors.

TRPV1 is implicated in several pain conditions, including inflammation, cancer-related pain, and neuropathic pain. Specifically, TRPV1 has been reported to play a role in orofacial pain conditions such as tooth pulp inflammation, temporomandibular disorders (TMD), oral cancer, and injury-induced pain from inferior alveolar nerve (IAN) damage. These findings indicate that TRPV1 may play a significant role in neuropathic pain, particularly trigeminal neuropathic pain.

The activation and sensitization of nociceptors can occur through both direct and indirect pathways. TRPV1 can be directly activated by increases in temperature or by chemical mediators released by immune cells such as resident mast cells (e.g. histamine) and polymorphonuclear leukocytes (PMNLs), as well as epithelial cells, Schwann cells, fibroblasts, and sympathetic post-ganglionic neurons (SPGN). Cross-talk between signaling pathways plays a crucial role in TRPV1 modulation. For example, TRPV1 can be activated or sensitized by the phospholipase C-mediated release of inositol trisphosphate (IP3) from phosphatidylinositol 4,5-bisphosphate (PIP2). The combined actions of diacylglycerol (DAG) and calcium ions (Ca²⁺) released from intracellular stores can activate classical protein kinase C (PKC) isozymes, such as PKCδ. The elevation of intracellular Ca²⁺ also activates Ca²⁺/calmodulin-dependent protein kinase II (CaMKII), further sensitizing TRPV1.

Additionally, activation of adenylate cyclases via G-protein-coupled receptors (GPCRs) leads to protein kinase A (PKA) activation, which can sensitize TRPV1. Another pathway involves PKCε, which is activated through a cAMP-dependent guanine exchange factor (EPAC) mechanism. The EPAC-dependent pathway plays a prominent role in nociceptor sensitization, particularly during ongoing inflammation, and is thought to contribute to the tissue “memory” of previous injuries, which can prolong pain sensitivity.

### The Role of Muscarinic Receptors in Neuropathic Pain

First-generation antihistamines are known to exhibit antimuscarinic effects. Muscarinic receptors are also G-protein-coupled receptors involved in the parasympathetic nervous system, named for their increased sensitivity to muscarine, a compound found in certain. The muscarinic receptor family consists of five subtypes: M1, M2, M3, M4, and M5. These receptors are activated by the neurotransmitter acetylcholine. Similar to histamine receptors (H1, H2, and H3), muscarinic receptors M1, M3, and M5 are excitatory, coupled to Gq proteins, which activate phospholipase C (PLC) and protein kinase C (PKC). PLC produces the second messengers diacylglycerol (DAG) and inositol triphosphate (IP3), which increase intracellular calcium and PKC activation, leading to an excitatory cellular response. Conversely, M2 and M4 receptors are inhibitory, coupled to Gi proteins that inhibit adenylyl cyclase, reducing protein kinase A (PKA) activity and cyclic adenosine monophosphate (cAMP) levels, resulting in an inhibitory response.

Several studies suggest that the cholinergic system, particularly muscarinic receptors, is involved in neuropathic pain. Central muscarinic M2 receptors have been shown to modulate neuropathic pain induced by traumatic nerve injury, and activation of these receptors in the insular cortex has been found to reduce oxaliplatin-induced neuropathic pain in male rats. Electrophysiological studies further indicate that presynaptic muscarinic receptors in the dorsal root ganglion (DRG) and trigeminal ganglion modulate primary afferent input to spinal or medullary dorsal horn neurons. Recent studies using in vivo siRNA knockdown techniques in rats demonstrated that M2 and M4 receptors predominantly mediate antinociception, while M1 and M3 receptors showed little involvement. Notably, M2 and M4 receptors are expressed at primary afferent terminals, and the M4 subtype appears particularly relevant in chronic pain states, such as diabetic neuropathy, where its expression is upregulated.

Dimethindene, a first-generation antihistamine with antimuscarinic properties, is an antipruritic used both orally and topically. It exists as a racemic mixture, where the(S) (+)-dimethindene enantiomer acts as a potent M2-selective muscarinic receptor antagonist, while the (R)-(−)-enantiomer is the bioactive agent for histamine H1 receptor binding. Several H1-receptor antagonists, such as mequitazine, cyproheptadine, clemastine, diphenylpyraline, promethazine, homochlorcyclizine, and Trimeprazine, have also demonstrated high affinity for muscarinic receptors (Ki=5.0-38 nM).

A key aspect of trigeminal neuralgia (TN), particularly in classical or idiopathic cases, is nerve compression by blood vessels, such as the superior cerebellar artery. This compression results in focal demyelination of the trigeminal nerve, causing abnormal nerve signaling, including ephaptic transmission, in which impulses “jump” between neurons. This misfiring of the trigeminal nerve generates spontaneous pain. Recent studies have highlighted the involvement of the M1 receptor (M1R) in remyelination, positioning it as a potential therapeutic target for diseases like multiple sclerosis (MS). MS is driven by myelin destruction due to an aberrant inflammatory response and failure to remyelinate efficiently, as oligodendrocytes responsible for axonal myelination are lost. While oligodendrocyte precursor cells (OPCs) can migrate to lesion sites, they often fail to differentiate, resulting in sustained demyelination over time.

M1R antagonists, such as clemastine, a first-generation antihistamine, have been shown to promote OPC differentiation into oligodendrocytes, leading to remyelination. MS patients often suffer from neuropathic pain, including TN, due to demyelination. Although clemastine showed modest improvement in MS disease progression remyelination via M1R inhibition, potentially with agents like Trimeprazine (which has muscarinic activity) may offer sufficient remyelination to alleviate TN pain through similar mechanisms.

### Proposed Mechanism of Action (MOA) of Trimeprazine Tartrate in Trigeminal Neuralgia

Trigeminal neuralgia (TN) is a chronic neuropathic pain condition characterized by hypersensitivity, driven by both peripheral and central sensitization. The pathophysiology of TN is complex and multifactorial, involving numerous molecular and cellular mechanisms. Various therapeutic interventions have targeted these mechanisms, with mixed clinical outcomes. The current standard of care for TN, such as carbamazepine and oxcarbazepine, focuses on hyperactive sodium channels in trigeminal neurons of the peripheral nervous system (PNS). However, these treatments are often associated with significant side effects and waning efficacy, leaving many patients with persistent, debilitating pain.

Applicant has demonstrated Trimeprazine tartrate's effectiveness in alleviating pain in both post-herpetic neuralgia (PHN) and TN, with a lasting therapeutic effect. While the clinical observations are not from a randomized clinical trial and could be subject to placebo effects and other confounders, they offer a promising finding for this devastating facial pain condition.

TN, being a multifaceted condition, may not be optimally managed by therapies targeting single pathways, such as sodium channels. Current pharmacological treatments for TN are not disease-modifying and primarily offer symptomatic relief. Microvascular decompression (MVD) remains the only disease-modifying treatment, providing significant, long-term pain relief for patients with nerve compression. However, TN patients without nerve compression, or for whom MVD is unsuccessful, must rely on long-term drug therapy, often involving multiple medications with limited efficacy.

Like many first-generation antihistamines, Trimeprazine has a broad range of therapeutic applications, including pruritus relief, sedation, anti-emetic effects, and antitussive activity. Its diverse pharmacological effects are attributed to its central and peripheral antihistamine properties and “off-target” actions, including antimuscarinic, anti-α-adrenergic, and anti-serotonergic effects. It is proposed that Trimeprazine, a first-generation antihistamine capable of crossing the blood-brain barrier (BBB), alleviates TN pain by modulating multiple mechanisms involving both the central nervous system (CNS) and PNS. This unique polypharmacological activity positions Trimeprazine as a promising novel therapeutic agent for alleviating the pain of TN, through the modulation of the diverse molecular pathways underlying its complex pathophysiology.

Trimeprazine has been widely used as an anti-itch medication, and there are shared mechanisms between itch and pain, including those involving histamine. It is therefore proposed that the primary mechanism of action (MOA) for Trimeprazine in TN involves stabilizing the H1 receptor (H1R) in its inactive conformation. This would reduce excessive histamine stimulation of trigeminal afferents and neurons within the CNS (dorsal root ganglia [DRG], trigeminal ganglia [TG], spinal dorsal horn, and thalamus). This action likely occurs via the PLCβ3, PIP2, DAG, IP3, and PKC signaling cascades, which regulate ion channels such as TRPV1, reducing neuronal sensitization.

Multiple first-generation H1-receptor antihistamines have been researched as potential analgesics and have shown promise in pre-clinical and clinical models however the discovery of, and subsequent widespread use of non-steroidal anti-inflammatory drugs (NSAIDs) seems to have quelled the interest in antihistamines as analgesics. Confounding results from pre-clinical studies targeting H2 and H3 receptors, the discovery of the role of newly identified H4 receptors in pain will require further research, especially on the interplay between histamine receptors before antihistamines once again attract the attention of pharmaceutical companies' pain programs. We consider the clinical data on the effectiveness of Trimeprazine, albeit in a small number of TN patients, coupled with planned pre-clinical studies and clinical trials will provide valuable insights into the role of first-generation antihistamines in neuropathic pain.

The role of TRPV1 in nociception is central to our hypothesis as histamine signaling is closely coupled to TRPV1 activation in both itch and pain. TRPV1, involved in various neuropathic pain conditions (e.g., PHN, diabetic neuropathy, chemotherapy-induced pain), may also play a role in trigeminal neuropathic pain. TRPV1 can be activated or sensitized via the PLC-mediated release of IP3 from PIP2, with DAG and intracellular Ca2+ further activating PKC isozymes such as PKCδ, which phosphorylate TRPV1. This cascade, including the activation of Ca²⁺-calmodulin-dependent protein kinase II (CaMKII), leads to TRPV1 sensitization and the perception of pain.

Trimeprazine's modulation of these pathways (via PKC) may reduce the hyperexcitability of trigeminal neurons, alleviating TN pain. Targeting TRPV1 for pain and itch relief has shown promise, especially using capsaicin a desensitizing TRPV1 agonist with analgesic, anti-pruritic, and anti-inflammatory action. An 8% capsaicin patch is approved for use in PHN and painful diabetic neuropathy. The clinical effectiveness of capsaicin led to the development of multiple orally available TRPV1 antagonists and desensitizing agonists for a variety of TRPV1 mediated pain and itch conditions. However, no drugs were approved for clinical use due to unwanted side effects (hyperthermia) in Phase I trials and a lack of efficacy in PhII trials, resulting in a lack of further development by pharmaceutical companies. Interestingly, the focus has shifted away from optimizing for highly selective and potent TRPV1 antagonists to a so-called “softer” drug approach, modulating TRPV1 with molecules based on the capsaicin backbone but designed to be enzymatically modified in affected tissues, and applied topically to reduce local side-effects such as burning and systemic effects, especially hyperthermia.

Additionally, Trimeprazine's modulation of H1R may impact inflammatory processes. Tissue damage, injury, or infection (e.g., Herpes zoster) involving the trigeminal neurons triggers immune responses, including the activation of resident mast cells and the recruitment of polymorphonuclear leukocytes (PMNLs), epithelial cells, Schwann cells, fibroblasts, and sympathetic post-ganglionic neurons (SPGNs). Mast cells, in particular, release histamine, which binds to H1R in peripheral afferents, DRG, and TG, further sensitizing TRPV1 and exacerbating pain. Trimeprazine may reduce this histamine-driven sensitization by stabilizing H1R and modulating immune responses, contributing to overall pain reduction.

The ability of Trimeprazine to modulate pain through H1R may also involve the suppression of the NF-κB immune response transcription factor via PLC and PIP2 signaling pathways. This suppression would lead to reduced antigen presentation, decreased expression of pro-inflammatory cytokines, and downregulation of cell adhesion molecules and chemotactic factors. Additionally, by stabilizing calcium levels, Trimeprazine may promote mast cell stability, further reducing histamine release. Together, these actions reduce both neuron sensitization and peripheral inflammatory processes, which ultimately dampen CNS sensitization and pain.

The off-target effects of Trimeprazine on muscarinic receptors may further enhance its analgesic profile. Both the M2 and M4 muscarinic receptor subtypes, which mediate antinociception, are expressed at primary afferent terminals. The M4 receptor is of particular interest in chronic pain conditions, as its expression increases in neuropathic pain states such as diabetic neuropathy.

Trimeprazine's high affinity for muscarinic receptors, shared with other H1 receptor antagonists, may contribute to its pain-relieving effects in TN.

In Applicant's work on a subset of TN patients treated with Trimeprazine, two out of seven have experienced long-term pain relief, with no recurrence of symptoms for over 12 months. This suggests a potential disease-modifying effect, possibly mediated by Trimeprazine's impact on inflammatory pathways. Additionally, recent studies have highlighted the role of the M1 muscarinic receptor in remyelination, which may be particularly relevant in TN cases involving nerve compression and demyelination. Trimeprazine's potential to promote remyelination via M1 receptor modulation could provide long-term, potentially disease modifying therapeutic benefits.

In conclusion, while the precise mechanism of action of Trimeprazine in TN remains to be fully elucidated, the current hypotheses suggest that because of its polypharmacology the drug acts via multiple pathways to reduce pain and potentially modify disease progression. Ongoing research aims to further characterize these mechanisms as part of efforts to repurpose and reformulate Trimeprazine for TN. Early clinical observations indicate that Trimeprazine offers significant pain relief in TN patients.

### EXAMPLES

Although the present invention has been described in considerable detail with reference to certain aspects thereof, other versions are possible. Therefore, the spirit and scope of the appended claims should not be limited to the description and the versions exemplified within this specification. Various aspects of the present invention will be illustrated with reference to the following non-limiting example.

The following table is a brief summary of the examples, describing seven patients suffering from TN along with their clinical outcome and comments related to each patient. In each case, administration of trimeprazine reduced or relieved the patients' pain, as described further in the examples.

| | TN | | |
|---|---|---|---|
| | Diagnosis | Clinical Outcome | Comment |
| Patient | Type 1 | Relief, no additional | Pain gone day 3 |
| 1 | | meds required | |
| Patient | Type 1 | Relief, no additional | 3 subsequent attacks |
| 2 | | meds required | aborted |
| Patient | Type 1 | Relief, no additional | “So nice to be pain |
| 3 | | meds required | free.” |
| Patient | Type 1 | Relief, no additional | Facial pain gone |
| 4 | | meds required | |
| Patient | Type 2 | Better, in addition to | Better and continuing |
| 5 | | gabapentin | Gabapentin |
| Patient | Type 2 | Better, can travel | After a year could leave |
| 6 | | | home |
| Patient | Type 2 | Better, no additional | “No bother now” |
| 7 | | meds required | |

### Example 1

A middle-aged male patient suffers from classic trigeminal (Type 1) neuralgia presenting with sharp, lancinating, severe pain in his left face precipitated by showering, eating, facial animation, touch of his facial skin and nose, and brushing his teeth. The episodes of pain are initiated by the stimuli above and immediately stop all other activity until they subside in several seconds. His behavior is modified to avoid the stimuli above, even affecting how he speaks and eats. He has had an MRI in the past that revealed there was no vascular compression basis for the diagnosis. His daily activities are dominated by fear of inducing another attack.

He does not take carbamazepine (Tegretol) for fear that he will be disqualified from holding his pilot's license. He does not take any analgesics. He had no remedy for these terrible pains before treatment with Trimeprazine.

The patient began treatment with Trimeprazine 5 mg morning and evening. The second day of the course the intensity and frequency of the attacks were in decline. After four days of medication all pain was gone. Since then, he has noted some discomfort in his face but not the sharp, almost unbearable pain of before. This has been successfully alleviated with from ½ to 1 pill of 5 mg taken once or twice a day. On most days he takes no medication now. There have been no side-effects other than drowsiness after taking the medication. He notes that this is a common side-effect for him to experience if a medication has any potential for causing drowsiness.

### Example 2

Patient, a 76-year-old male, had a history of Lyme disease affecting the V1 and V2 distributions of the right trigeminal nerve. The disease had caused cutaneous anesthesia in the right forehead and cheek. This resolved with successful antibiotic therapy. Beginning at about 19 years after the bout of Lyme disease he noted sharp lightning-like pains in his right cheek. Brief and usually during cutaneous stimulation, like being in a shower. There were perhaps one or two of these occurrences per year. At 22 years after the initial Lyme disease, he experienced a severe attack of Trigeminal Neuralgia (TN) affecting his right cheek (Type 1). There were on average 8 bouts of severe stabbing pain per day precipitated by facial animation, touching, opening his mouth to eat, brushing his teeth, and even a gust of cold wind. He treated himself with doxycycline, fearing a reactivation of Lyme disease. His attacks disappeared after 14 days, and he was asymptomatic. He obtained Trimeprazine and when sharp pains recurred at seven and eleven months after the TN attack, he took 5 mg twice a day and managed to immediately stop all recurring pains. There have been no recurrent attacks of TN in the 12 months since the last treatment with Trimeprazine.

### Example 3

Patient is an elderly male with TN (Type 1) unsuccessfully controlled with carbamazepine and gabapentin. He was quite miserable, being unable to even brush his teeth without precipitating a severe bout of pain. He was begun on Trimeprazine at 5 mg twice a day and except for some drowsiness, tolerated the medicine well. The pain began to recede and by ten days after starting treatment he was essentially pain free and able to eat, speak, and brush his teeth without fear of precipitating an acute bout of TN pain. This relief has been enduring. He discontinued the carbamazepine and gabapentin.

### Example 4

Patient is a 77 year old male who suffers from TN which began 3 years ago after a dental procedure. The TN was manifest by sharp, lancinating pain in the left side of his jaw (Type. 1). These periods of pain were episodic, precipitated by stimuli like brushing his teeth, at least once per day, with periods of vulnerability lasting 3-4 weeks each occurring 3-4 times per year. His mother had suffered with the same pattern of TN. When started on Trimeprazine he was also taking Pregabalin. After a first course of 10 days of Trimeprazine 5 mg BID his jaw pain was gone but he had a sensitive area on the side of his head above his ear. This disappeared entirely with a second course of Trimeprazine 5 mg BID for several days.

### Example 5

Patient is a young woman who developed severe ophthalmic herpes zoster involving the right side of her face and her right eye. She had extensive ocular involvement requiring medications for a full year. Her corneal difficulties were accompanied by a severe lingering neuralgia in the affected ophthalmic distribution of the trigeminal nerve (Type 2), which plagued her every waking hour. She was on 12 medications including carbamazepine 200 mg BID and pregabalin 150 mg TID, ophthalmic medications, and oxycodone as needed for pain relief. She began trimeprazine and noted some improvement by 5 days after starting the medication and then asked for more to continue the treatment. Four months or so after, she reported “I'm probably at a max pain of 5 and average 2, which is wonderful!” She has had persistent facial pain in the area of her ophthalmic branch of the trigeminal nerve, but it has been less. She is contemplating starting a second course of trimeprazine at 10 mg BID, twice the amount of the first dose she took.

### Example 6

This patient suffered from shingles on her forehead which was followed by lingering neuralgia in the affected ophthalmic distribution of the trigeminal nerve. She had persistent pain (TN Type 2) for about six months, though not as severe as Patient 5. This patient was able to function in a normal way. By 6 days after starting trimeprazine at 5 mg BID she reported “Yes, on medicine and it does help my head. Only have a few pills left. It is a relief not to have a headache!” She has not had a second course of medication. She has some residual pain from time to time that is well controlled with Tylenol.

### Example 7

A representative clinical protocol for the treatment of trigeminal neuralgia is outlined herein.

Number of patients: 80-100.

Study duration: 12-18 months (12 week treatment period).

Study design: Randomized double blind (DB) withdrawal studies, comparison to standard of care. The study may include a screening period, a 7-day run-in period, a 4 or 6-week single-dose-blind dose-optimization period, or a screening period, 7-day run-in period, 4-week open-label period, and 14-week double-blind period. There is a potential for a long-term extension where applicable for patients completing the double-blind period.

Primary endpoint: proportion of participants classified as responders at Week 12 of the double-blind period.

Secondary endpoints: safety measures, quality of life, and evaluation of drug population pharmacokinetics.

Number of sites: 10

### Primary Outcome Measure: Frequency and Duration of Pain Attacks as Recorded in a Pain Diary

Secondary outcome measure: Penn-FPS-R Quality of Life measure, PGIC, number of times patient used “rescue-medication” (e.g. existing SOC drugs, opioids, etc.).

Inclusion criteria: ≥18 years old who have classical, purely paroxysmal TN diagnosed ≥3 months prior to study entry, who experience ≥3 paroxysms of pain/day.

Endpoints of trials: based upon Patient Reported Outcome measures using the frequency and duration of pain attacks as recorded in a pain diary, Patient Global Impression of Change (PGIC) score and the Penn-Facial-Pain-Score-Revised, EuroQoL 5-Dimensions 5-Level version (EQ-5D-5L) score, and Work Productivity and Activity Impairment (WPAI). Change in use of “rescue medication” in each arm before and after the treatment period.

Formulations of Trimeprazine: 5 mg BID Trimeprazine sulfate or Trimeprazine tartrate. For most patients, dosing will be 1×5 mg oral thin film (OTF) twice per day, morning and evening. Alternative dosing for elderly patients (especially those with symptoms of dizziness) may use titration, starting with a 1×2.5 mg OTF or 1×OTF tablet once per day in the evening to manage potential side effects of dizziness.

Treatment may be stopped based on reduction in the intensity and frequency of pain, e.g. in the case of TN, patients may cease treatment once the frequency and intensity of paroxysms are reduced to zero, restarting treatment if paroxysms start again. However, in the case of other conditions which manifest as more continuous pain, e.g. PHN, treatment would likely continue for the long term. Depending on the indication, drug could be used prophylactically to manage the intensity and frequency of pain, especially as it may act at the sensory ganglia involved in the perception of pain in the Dorsal Root Ganglion (PHN, CIPN, DNP), or Gasserian ganglion (TN), without wishing to be bound by theory.

### Example 8

A representative clinical protocol for the treatment of post-herpetic neuralgia is outlined herein.

Postherpetic neuralgia (PHN) is the most common long-term complication of varicella-zoster virus (VZV) reactivation, also known as human herpesvirus-3 (HHV-3). This reactivation of dormant VZV is known as herpes zoster or shingles. VZV is the causative agent for the childhood condition varicella, colloquially known as chickenpox.

The hallmark of PHN is a lancinating/burning pain in a unilateral dermatomal pattern that persists for three or more months after the onset of a herpes zoster (HZ) outbreak.

Postherpetic neuralgia occurs in a subset of the population suffering from an episode of acute HZ. Well-established risk factors for an acute HZ episode progressing to PHN include age, severe immunosuppression, the presence of a prodromal phase, severe pain during zoster outbreak, allodynia, ophthalmic involvement, and diabetes mellitus.

Persistent (more than or equal to 3 months) lancinating/burning pain, allodynia, paresthesias, pruritus, dysesthesias, and/or hyperalgesia at or near the area of the rash is characteristic of PHN. PHN also interferes with patients' quality of life, and disturbed sleep is a prevalent complaint. Pain-associated sleep interference in turn enhances pain and/or reduces pain tolerance.

Treatment endpoints would be reduction in overall intensity and/or elimination of PHN pain, and improved sleep (less PHN pain-related sleep disturbance). Pain is described as lancinating/burning pain, allodynia, paresthesias, pruritus, dysesthesias, and/or hyperalgesia at or near the area of the HZ rash. Sleep is also affected.

Number of patients: 100-120.

Study duration: 12-18 months (12 week treatment period).

Study Design: Randomized, Double Blind, Placebo Controlled, efficacy study. Treatment arm: Drug at 5 mg dose, twice daily, for the treatment period. Placebo comparator: Placebo (sugar pill) twice daily for the treatment period.

Primary endpoint: Change from Baseline in average daily pain intensity score for the previous 7 days (Week 8 or Final visit).

Secondary endpoints: pain scales, sleep score, quality of life, profile of mood states.

Number of sites: 20.

Countries: US only.

Primary outcome measure: Basic Pain Inventory (BPI) (PHN version).

Secondary outcome measure: BPI, McGill Short Form pain scale, Clinician and Patient Global Impression of Change, EuroQoL 5-Dimensions 5-Level version (EQ-5D-5L) score, Sleep diary, Mood, number of times patient used “rescue-medication” (e.g., existing SOC drugs, opioids, etc.).

Inclusion criteria: ≥50 years old, male and female subjects with postherpetic neuralgia whose pain has been present for >3 months following healing of the herpes zoster rash. Female subjects are not of child-bearing potential (e.g., sterilized, postmenopausal).

Endpoints of the trials would be based upon reduction in pain (based on the BPI score) compared to baseline at the week 8 or final visit. Additional secondary endpoints would be based on reduction in pain compared to baseline at each visit, improvements compared to baseline in: Clinician and Patient Global Impression of Change scores, EuroQoL 5-Dimensions 5-Level version (EQ-5D-5L) scores, Mood scores, and sleep scores. Change in use of “rescue medication” in each arm before and after the treatment period.

### Example 9

A representative clinical protocol for the treatment of diabetic neuropathic pain is outlined herein.

Diabetic neuropathy is the most common complication of diabetes mellitus (DM), affecting as many as 50% of patients with type 1 and type 2 DM. Diabetic neuropathic pain (DNP) is clinically defined as pain resulting from peripheral, autonomic, focal or proximal nerve damage in patients with diabetes. It most commonly manifests distally to affect the hands and feet and can occur in patients with either a type 1 (TID) or type 2 diabetes (T2D) diagnosis. The prevalence of painful neuropathy in Type 2 diabetes is more than twice that seen in Type 1 diabetes.

Most patients experience moderate-to-severe pain with the majority of patients finding it difficult to express the character of the pain. The common neuropathic pain descriptors such as ‘burning’, ‘electric shocks’, ‘shooting/stabbing down the leg’, ‘pins and needles and tingling’, and ‘numbness’ can be elicited on further questioning. Patients may also have features of evoked pain such as allodynia (painful response to non-painful stimuli) and hyperalgesia (increased pain from a stimulus that normally provokes pain), which could have a significant impact on everyday activities. Pain is usually present in the feet, and this may ascend to involve the lower limbs and occasionally both hands. Pain typically worsens during the night resulting in sleep disturbance and tiredness.

Pain in combination with physical disability because of other long-standing complications of diabetes substantially impairs the quality of life. Patients with neuropathic pain have markedly lower scores on quality-of-life domains, including enjoyment of life, sleep, physical mobility, self-care, and energy levels.

Treatment endpoints would be reduction in the overall pain of DNP, reduction in sleep disturbance together with improvements in quality of life and mood scores.

Number of patients: 200+.

Study duration: 12-18 months (12 week treatment period).

Study Design: Randomized, Double-Blind, Placebo-Controlled, Parallel Group, efficacy study.

Treatment arm: Drug at 5 mg dose, twice daily, for the treatment period.

Placebo comparator: Placebo (sugar pill) twice daily for the treatment period.

Primary endpoint: Change from Baseline (Day 7-Day 1) in mean average daily pain intensity score compared to the mean average pain intensity score for the Final week (Week 12).

Secondary endpoints: pain scales, sleep score, quality of life, profile of mood states.

Number of sites: 20.

Countries: US only.

Primary outcome measurement: 11-point pain intensity numerical rating scale (PI-NRS).

Secondary outcome measurement: PI-NRS, Clinician and Patient Global Impression of Change, EuroQoL 5-Dimensions 5-Level version (EQ-5D-5L) score, Sleep diary, Mood.

Inclusion criteria: >18 and <80 years old, male and female subjects Type 1 or Type 2 diabetes of at least 6 months with optimized and stable glycemic control during the 3 months prior to Screening. Douleur Neuropathique 4 (DN4) score of >=4, number of times patient used “rescue-medication” (e.g., existing SOC drugs, opioids, etc).

Endpoints of the trials would be based upon reduction in pain (based on the PI-NRS) compared to baseline at the week 8 or final visit. Additional secondary endpoints would be based on reduction in pain compared to baseline at each visit (or patient self-report), improvements compared to baseline in: Clinician and Patient Global Impression of Change scores, EuroQoL 5-Dimensions 5-Level version (EQ-5D-5L) scores, Mood scores, and sleep scores. Change in use of “rescue medication” in each arm before and after the treatment period.

### Example 10

A representative clinical protocol for the treatment of chemotherapy-induced neuropathic pain is outlined herein.

Chemotherapy-induced neuropathic pain (CINP) is one of the most severe side effects of anticancer agents, such as platinum- and taxanes-derived drugs (oxaliplatin, cisplatin, carboplatin and paclitaxel), and is essentially caused by injury to the somatosensory nervous system after anticancer drug treatment, and it is one of the major causes of neuropathic pain in clinical practice for cancer.

Sensory symptoms usually manifest as spontaneous or evoked abnormal sensations such as paraesthesia, dysesthesias, numbness, burning, shooting or electric shock sensations, as well as allodynia or hyperalgesia evoked by mechanical or thermal stimuli. The symptoms usually affect the extremities of the upper and lower limbs (“stocking and glove” distribution) and progress to the proximal regions of the body.

Chemotherapy-induced neuropathic pain manifests initially as an acute pain syndrome, with sensory symptoms arising during or just after drug administration, progressing to a chronic neuropathy after repetitive treatment cycles.

However, acute neuropathy generally subsides between treatments, while chronic neuropathy can persist for months or years, with 47% of patients treated with anti-cancer drugs still experiencing peripheral neuropathy symptoms after 6 years of treatment termination. The chronic pain of CINP severely impairs the quality of life of cancer patients.

CINP patients report paraesthesia, dysesthesias, numbness, burning, shooting or electric shock sensations, as well as allodynia or hyperalgesia evoked by mechanical or thermal stimuli, that affect the extremities of the upper and lower limbs (“stocking and glove” distribution) and progress to the proximal regions of the body.

Treatment endpoints would be reduction in the overall pain of CINP, together with Quality of Life measures.

Number of patients: 25-30.

Study Design: Randomized, Double-Blind, Placebo-Controlled, Parallel Group, efficacy study.

Treatment arm: Drug at 5 mg dose, twice daily, for the treatment period.

Placebo comparator: Placebo (sugar pill) twice daily for the treatment period.

Primary endpoint: Compare patient-reported pain intensity scores after the treatment period (12 weeks) for the two arms of the trial.

Secondary endpoints: pain scale (NPSI), cancer related symptom measures (e.g. FACT-taxane), change in user of “rescue medication”.

Number of sites: up to 10.

Countries: US only.

Primary outcome management: Basic Pain Inventory (BPI).

Secondary outcome measurement: Neuropathic Pain Symptom Inventory (NPSI), FACT-Taxane, FACT-GOG-NTX or appropriate FACT instrument for chemotherapy used, number of times patient used “rescue-medication” (e.g., existing SOC drugs, opioids, etc.).

Inclusion criteria: Patients will be at least 18 years of age. Patients will be experiencing moderate to severe peripheral neuropathic pain. Patients must have chronic peripheral neuropathic pain will be defined as pain of 3 or more months duration which began in association with chemotherapy. Patient's will have bilateral peripheral neuropathic pain symptoms primarily involving the feet. Patients must have cancer (any stage).

Endpoints of the trials would be based upon reduction in pain (based on the BPI score) at the end of treatment (Week 12). Additional secondary endpoints would be based on reduction in pain (using NPSI, FACT-XXX) compared to baseline at the end of the treatment period in the arms of the trial. Change in use of “rescue medication” in each arm before and after the treatment period.

### Example 11

### PhI/II Bioavailability, Safety and Efficacy Study of Trimeprazine for the Treatment of Trigeminal Neuralgia

The typical or “classic” form of the disorder (called “Type 1” or TN1) causes extreme, sporadic, sudden burning or shock-like facial pain that lasts anywhere from a few seconds to as long as two minutes per episode. These attacks can occur in quick succession, in volleys lasting as long as two hours.

The “atypical” form of the disorder (called “Type 2” or TN2), is characterized by constant aching, burning, stabbing pain of somewhat lower intensity than Type 1.

Both forms of pain may occur in the same person, sometimes at the same time. The intensity of pain can be physically and mentally incapacitating.

The trigeminal nerve is one of 12 pairs of nerves that are attached to the brain. The nerve has three branches emanating from the “Gasserian Ganglion” (semilunar ganglion) that conduct sensations from the upper, middle, and lower portions of the face, as well as the oral cavity, to the brain. The ophthalmic, or upper, branch supplies sensation to most of the scalp, forehead, and front of the head. The maxillary, or middle, branch stimulates the cheek, upper jaw, top lip, teeth and gums, and to the side of the nose. The mandibular, or lower, branch supplies nerves to the lower jaw, teeth and gums, and bottom lip. More than one nerve branch can be affected by the disorder. Rarely, both sides of the face may be affected at different times in an individual, or even more rarely at the same time (called bilateral TN).

Pain varies, depending on the type of TN, and may range from sudden, severe, and stabbing to a more constant, aching, burning sensation. The intense flashes of pain (paraxosyms) can be triggered by vibration or contact with the cheek (such as when shaving, washing the face, or applying makeup), brushing teeth, eating, drinking, talking, or being exposed to the wind. The pain may affect a small area of the face or may spread. Bouts of pain rarely occurs at night, when the affected individual is sleeping. TN is typified by attacks that stop for a period of time and then return, but the condition can be progressive. The attacks often worsen over time, with fewer and shorter pain-free periods before they recur. Eventually, the pain-free intervals disappear and medication to control the pain becomes less effective.

### Example 12

Study Design Synopsis: The study is designed as a Phase I (b)/II (b) bioequivalence and proof-of-concept efficacy study to assess bioequivalence, formulation, safety, dosing in healthy volunteers and efficacy of Trimeprazine in TN Patients with a suboptimal response to their current anti-pain therapy. Ph1 (b)—part A will assess the bioequivalence and safety of the tablet form of Trimeprazine and the Oral Thin Film (OTF) form of Trimeprazine in healthy volunteers. Ph1 (b)—part B will be an open-label, multi-ascending dose study of the OTF form of Trimeprazine in healthy volunteers to establish the maximum tolerated dose (MTD). The Phase II (b) study will assess efficacy of the OTF of Trimeprazine at the MTD in a randomized, controlled study compared to control in TN patients.

Phase 1 (b)—Part A

Primary Purpose: Bioequivalence/Safety of the tablet/Oral Thin Film (OTF) form in healthy volunteers

Allocation: Randomized, controlled, crossover study comparing the tablet form of the drug and the OTF

Interventional Model: Random, Cross-over
Trimeprazine tablet
Trimeprazine OTF

Masking: None—open-label

Subjects: n=52 (total for part A and part B)

| Participant Group/Arm | Intervention/Treatment |
|---|---|
| Phase I(b) Part A: Experimental: | Drug: Trimeprazine |
| Trimeprazine: | On Day 1 of each treatment period, |
| Normal healthy volunteers (NHV) will be | participants will receive three daily oral |
| assigned randomly to one of the two | doses (after morning and evening meals, |
| bioequivalence cross-over sequences | and at bedtime) of Trimeprazine, |
| (sequence AB or sequence BA) | according to the assigned sequence: |
| | Treatment A: a three-times daily |
| | oral dose of 5 mg of Trimeprazine |
| | (reference tablet formulation, |
| | 3 × 5.0 mg tablets). |
| | Treatment B: three-times daily oral |
| | dose of 5 mg of Trimeprazine (test |
| | OTF formulation, 3 × 5.0 mg OTF). |
| | The treatment period will be 3 days, |
| | washout period between sequences will be |
| | 4 days. |

Phase 1 (b)—Part B

Primary Purpose: Establish the Maximum Tolerated Dose (MTD) of the OTF form of Trimeprazine healthy volunteers

Allocation: Open label, multi-ascending dose of the OTF

Interventional Model: Parallel Assignment?
Trimeprazine OTF
Masking: None, open-label

| Participant Group/Arm | Intervention/Treatment |
|---|---|
| Phase I(b) Part B: Experimental: | Drug: Trimeprazine |
| Trimeprazine: | Participants will receive three daily oral |
| Normal Healthy Volunteers will receive | doses (up to the max dose for that dose |
| thrice daily dose of Trimeprazine in OTF | regimen) of Trimeprazine OTF in a |
| form | multiple ascending dose regimen. |
| | Trimeprazine dose will be titrated over 16 |
| | days; dose escalations (5.0 mg, 10.0 mg, 15 |
| | and 20.0 mg) will be separated by 4 days, |
| | with the final dose administered for a |
| | minimum of 14 days, up to a maximum of |
| | the end of the 12 week treatment period. |
| | The minimum starting dose will be 5.0 mg, |
| | which can be titrated up to a maximum dose |
| | of 20.0 mg, which is the current maximum |
| | dose of Trimeprazine for its original |
| | indication |

### Phase II (b)

Primary Purpose: Proof of concept study to determine the efficacy of the OTF form of Trimeprazine in TN patients.

Allocation: Randomized, controlled comparing OTF of Trimeprazine with Placebo

Interventional Model: Parallel Assignment
Drug: Trimeprazine OTF
Drug: Placebo (matched to Trimeprazine OTF)

Masking: Double (Participant/Investigator)

Subjects: n=40

| Participant Group/Arm | Intervention/Treatment |
|---|---|
| Experimental: Trimeprazine | Drug: Trimeprazine OTF |
| Study participants with Trigeminal | Participants will receive twice-daily oral |
| Neuralgia (TN) will receive twice daily | Trimeprazine for the 12 week treatment |
| dose of Trimeprazine in OTF form | period |
| Placebo Comparator: Placebo: | Drug: Placebo OTF |
| Participants with TN will receive a twice- | Participants will receive a twice-daily oral |
| daily dose of matching placebo OTF. | matching placebo OTF for the 12 week |
| | treatment period. |

### Endpoints

### Primary Outcome

| Outcome Measure | Measure Description | Time Frame |
|---|---|---|
| PhI(b) Part A: Incidence and severity of | Monitor incidence of | Up to Day 22 |
| adverse events, laboratory, vital signs and | adverse events | |
| cardiovascular events. | | |
| PhI(b) Part A: Maximum observed | Plasma concentration of | 0 h pre-dose, 0.5, |
| concentration (Cmax) | drug | 1.0, 1.5, 2.0, |
| | | 2.5, 3, 6, 8, 12, |
| | | 24, 36, 48 post |
| | | dose |
| PhI(b) Part A: Time to reach Cmax (Tmax) | Time to reach | 0-48 h |
| | concentration of drug | |
| PhI(b) Part A: Area under the concentration- | Calculated measure | 0-48 h |
| time curve from time 0 to time of the last | | |
| measurable drug concentration (AUC0-t) | | |
| PhI(b) Part A: Area under the concentration- | Calculated measure | 0-48 h |
| time curve from time 0 extrapolated to | | |
| infinity (AUCinf) | | |
| PhI(b) Part A: Terminal elimination half-life | Measure urine levels of | Oh pre-dose, |
| (t½) | drug | 4, 8, 12, 24, |
| | | 48 post-dose |
| PhI(b) Part A: Apparent total body | Calculate | 0-48 h |
| clearance (CL/F) | | |
| PhI(b) Part B: Incidence and severity of | Monitor incidence of | Up to 12 weeks |
| adverse events. | adverse events | |
| Phase II(b): Reduction in the number of | Measure number of TN | Up to 12 weeks |
| daily pain attacks compared to baseline | attacks per day using | |
| | electronic pain diary | |
| Phase II(b): Reduction in the duration of | Measure duration of | Up to 12 weeks |
| daily pain attacks compared to baseline | each TN attack per day | |
| | using electronic pain | |
| | diary | |
| Phase II(b): Time to Loss of Efficacy or | To assess the duration | Up to 12 weeks |
| pain recurrence defined as the confirmed | of the effect on pain. | |
| increase in the number of weekly paroxysms | | |
| or re-emergence of continuous pain and/or | | |
| the need for rescue medication*. | | |
| *“rescue medication” is defined as the | | |
| patients current prescribed medication at | | |
| the prescribed dose and frequency | | |

### Secondary Outcome Measures

| | | Time |
|---|---|---|
| Outcome Measure | Measure Description | Frame |
| Phase II(b): Mean change in the total | Impact on facial pain measured | 12 |
| patient-rated Penn-Facial Pain Scale- | by patient rated scale via Penn- | weeks |
| Revised (Penn-FPS-R) scale compared to | FPS-R | |
| baseline | | |
| Phase II(b): Time between attacks | Time between each attack of | 12 |
| | pain as measured by patient | weeks |
| | electronic pain diary | |
| Phase II(b): Frequency of attacks | Impact on facial pain measured | 12 |
| (paroxysms). | by patient rated scale via Penn- | weeks |
| | FPS-R | |
| Phase II(b): Severity of attacks | Impact on facial pain measured | 12 |
| (paroxysms). | by patient rated scale via Penn- | weeks |
| | FPS-R | |
| Phase II(b): Severity of continuous pain. | Impact on facial pain measured | 12 |
| | by patient rated scale via Penn- | weeks |
| | FPS-R | |
| Phase II(b): Duration of continuous pain. | Impact on facial pain measured | 12 |
| | by patient rated scale via Penn- | weeks |
| | FPS-R | |
| Phase II(b): Change in Patient Global | Impact on facial pain. | 12 |
| Impression of Change (P-GIC). | | weeks |
| Phase II(b): Change in Medication | Impact on facial pain. | 12 |
| Satisfaction Questionnaire (MSQ). | | weeks |

### Other Outcome Measures

| | | Time |
|---|---|---|
| Outcome Measure | Measure Description | Frame |
| Phase II(b): Mean change | Evaluate 12-week of up to thrice daily | 12 weeks |
| from baseline to Week 12 in | dosing of Trimeprazine on pain associated | |
| the total patient-rated Penn- | with TN on the following: | |
| Facial Pain Scale-Revised | Impact on Facial Pain | |
| (Penn-FPS-R) scale. | Patient perceived change of the | |
| | pain | |
| | Pain assessments | |
| | Pain freedom | |
| | Patient medication satisfaction | |
| Phase II(b): Measure Patient | Evaluate 12-week of up to thrice daily | 12 weeks |
| Global Impression of change | dosing of Trimeprazine on pain associated | |
| from baseline to Week 8. | with TN on the following: | |
| | Impact on Facial Pain | |
| | Patient perceived change of the | |
| | pain | |
| | Pain assessments | |
| | Pain freedom | |
| | Patient medication satisfaction | |
| Phase II(b): Number and | Evaluate 12-week of up to thrice daily | 12 weeks |
| severity of attacks | dosing of Trimeprazine on pain associated | |
| (paroxysms). | with TN on the following: | |
| | Impact on Facial Pain | |
| | Patient perceived change of the | |
| | pain | |
| | Pain assessments | |
| | Pain freedom | |
| | Patient medication satisfaction | |
| Phase II(b): Duration of | Evaluate 12-week of up to thrice daily | 12 weeks |
| continuous pain compared | dosing of Trimeprazine on pain associated | |
| with baseline | with TN on the following as measured by | |
| | patient rated scale via Penn-FPS-R: | |
| | Impact on Facial Pain | |
| | Patient perceived change of the pain | |
| | Pain assessments | |
| | Pain freedom | |
| | Patient medication satisfaction | |
| Phase II(b): Severity of | Evaluate 12-week of up to thrice daily | 12 weeks |
| continuous pain compared | dosing of Trimeprazine on pain associated | |
| with baseline. | with TN on the following as measured by | |
| | patient rated scale via Penn-FPS-R: | |
| | Impact on Facial Pain | |
| | Patient perceived change of the pain | |
| | Pain assessments | |
| | Pain freedom | |
| | Patient medication satisfaction | |
| Phase II(b): Number of pain | Evaluate 12-week of up to thrice daily | 12 weeks |
| free days. | dosing of Trimeprazine on pain associated | |
| | with TN on the following: | |
| | Impact on Facial Pain | |
| | Patient perceived change of the pain | |
| | Pain assessments | |
| | Pain freedom | |
| | Patient medication satisfaction | |
| Phase II(b): Patient reported | Evaluate 12-week of up to thrice daily | 12 weeks |
| rating of the Medication | dosing of Trimeprazine on pain associated | |
| Satisfaction Questionnaire | with TN on the following: | |
| (MSQ). | Impact on Facial Pain | |
| | Patient perceived change of the pain | |
| | Pain assessments | |
| | Pain freedom | |
| | Patient medication satisfaction | |
| Phase II(b): The impact of | | 12 weeks |
| pain on general activities of | | |
| daily living captured in patient | | |
| diary cards. | | |
| Phase II(b): Assess the level | Evaluate the level of depression over the | 12 eeks |
| of depression using the Beck | treatment period | |
| Depression Inventory- II (BDI- | | |
| II) | | |

### Description of Study Cohorts

### Healthy Cohort

### Inclusion Criteria Summary

1. Ability and willingness to provide written informed consent and to comply with the study procedures.
2. Fluency in the language of the investigator, study staff and the informed consent
3. Age 45-75 years (similar age range to patients with TN)
4. All subjects must practice effective contraception during the study and be willing and able to continue contraception for 90 days after the administration of study treatment.
5. Must be in good health as determined by the Investigator, based on medical history and screening evaluations.

### Exclusion Criteria

1. History of any clinically significant cardiac, endocrine, gastrointestinal, hematologic, hepatic, immunologic, metabolic, urologic, pulmonary, neurologic, dermatologic, psychiatric, or renal disease, or other major disease, as determined by the Investigator.
2. Previous exposure to Trimeprazine
3. Other unspecified reasons that, in the opinion of the Investigator or Sponsor, make the subject unsuitable for enrollment.

### Trigeminal Neuralgia Cohort

### Inclusion Criteria Summary:

1. Ability and willingness to provide written informed consent and to comply with the study procedures.
2. Fluency in the language of the investigator, study staff and the informed consent.
3. Age 18-75 years.
4. Diagnosis of primary trigeminal neuralgia (TN) as per the ICHD3 criteria confirmed by the study neurologist.
5. Experience pain due to TN and at baseline, experience at least 3 paroxysms per day of at least intensity of 4 or more on a pain intensity numerical rating scale (PI-NRS) during the last 7 days.
6. Female patients who are either sterile or menopausal. For female patients with childbearing potential, must be neither pregnant nor lactating (with appropriate contraceptive precautions and prior negative pregnancy tests).

### Exclusion Criteria Summary:

Patients who meet any of the following criteria will be excluded from participation in this study:
1. Current or prior history of any major psychiatric diagnoses unrelated to TN. Patients with TN-related depressive symptoms are permitted.
2. Current or prior history of mania, or psychotic episodes.
3. History of DSM-5-defined substance dependence (Diagnostic and Statistical Manual for Mental Disorders, 5th edition) and/or substance abuse in the last six months [180 days], except for nicotine.
4. Known allergic reaction to the investigational drug or one of its components.
5. Patients with secondary TN as per the ICHD3 criteria.
   Medication history:
6. Previous treatment with Trimeprazine, except with the prior agreement of the medical monitor.
7. Treatment with antipsychotics within six months (180 days) of screening.
8. Any investigational drug within 90 days prior to initiation of study drug.
   Medical status:
9. Evidence of clinically significant, uncontrolled, unstable medical conditions or recently diagnosed cardiovascular disease, such as ischemic heart disease, coronary artery vasospasm, and cerebral ischemia. Subjects with myocardial infarction, acute coronary syndrome, percutaneous coronary intervention, cardiac surgery, stroke or transient ischemic attack during the 6 months prior to screening.
10. Subject has a history of gastric, or small intestinal surgery (including gastric bypass, gastric banding, gastric sleeve, gastric balloon, etc.) that may, in the opinion of the investigator, may cause malabsorption, or has a disease of the GI tract that causes malabsorption.
11. Body mass index>39 kg/m²

### Power Calculation

Power calculations in pain trials are notoriously hard to perform and depend on the design of the study.

However in TN, specifically Type 1 (which presents as multiple daily episodes of lancinating pain attacks), we have chosen the primary endpoint of reduction in the number of daily attacks. Most patients have an average of 7-10 of these pain attacks per day, and a 30% reduction (i.e. 5-7 per day from 7-10) to a 50% reduction (i.e. 3-5 per day from 7-10) in the number of those attacks would be clinically significant for the patient.

Therefore, calculating the number of participants uses the following parameters. We assume two independent study groups from the parallel assignment (treatment vs. placebo) and we consider a clinically significant result of the drug to be a 30% reduction in pain attacks per day, i.e. a reduction of 3 compared to baseline.

n=16 participants per group and with a 10% dropout rate=16/(1-0.1)=17

A total of 35 participants are needed in total to power the trial.

### Example 13

Pharmaceutical formulation: quality target product profile (QTPP).

A formulation of Trimeprazine has been evaluated in an in vitro model of the buccal cavity. The quality target profile for this formulation is disclosed in the following tables.

| QTPP Elements | Target |
|---|---|
| Dosage Form | Sublingual oral thin film |
| Dosage Design | Monolayer thin film system |
| Route of | Sublingual delivery |
| administration | |
| Strengths | Equivalent 5 mg tablet (priority) and Potential |
| | for 2.5 mg or 10 mg |
| Pharmacokinetics | To match adult 5 mg oral tablet dose: |
| | Dose rate† _(Rate in) = Cₘₐₓ * CL (Rate out) = |
| | 607-1089 μg/h FB to match 5 mg tablet if reported |
| | concentration is FB, or |
| | 484-869 μg/h kg FB to match 5 mg tablet if |
| | reported concentration is salt. |
| | Flux requirement with max film size of 6 cm² and |
| | bi-directional delivery: |
| | >51-91 μg/cm²/h FB to match 5 mg tablet if |
| | reported concentration is FB, or |
| | >40-72 μg/cm²/h FB to match 5 mg tablet if |
| | reported concentration is salt. |
| | Lag time: comparable to oral tablets |
| Stability | 2-year shelf life or longer |
| Storage | Ambient condition |

| | | |
|---|---|---|
| Drug | Description | Trimeprazine tartrate sublingual oral |
| Product | | thin film |
| Quality | Unit Dimensions | TBD (≤6 cm²) |
| Attributes | Identification | Print on the film |
| | Assay | 90-110% of LC |
| | Content | The acceptance value (AV) should be |
| | Uniformity | less than or equal to 15.0 |
| | Water Content | <0.75 A_{w} |
| | Disintegration | TBD |
| | Time | |
| | Related | ICH Q3b guideline |
| | substances | |
| | (HPLC) | |
| | Residual | ICH Q3c guideline |
| | Solvents | |
| | Heavy metals | ICH Q3d guideline |
| | Mucoadhesion | TBD. Must adhere to mucosa for drug |
| | | delivery |
| | Tensile Strength | TBD |
| | Microbial Limits | Total Aerobic Microbial Count (cfu/g or |
| | | cfu/ml): <10² |
| | | Total Combined Yeasts/Molds Count |
| | | (cfu/g or cfu/ml): <10¹ |
| | | Absence of Staphylococcus aureus |
| | | Absence of Pseudomonas aeruginosa |
| | Mucosal | No mucosal irritation and sensitization |
| | Irritation and | |
| | Sensitization | |

This disclosure is not limited to the particular systems, devices and methods described, as these may vary. The terminology used in the description is for the purpose of describing the particular versions or embodiments only and is not intended to limit the scope.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 compounds refers to groups having 1, 2, or 3 compounds. Similarly, a group having 1-5 cells refers to groups having 1, 2, 3, 4, or 5 compounds, and so forth.

Various of the above-disclosed and other features and functions, or alternatives thereof, may be combined into many other different systems or applications. Various presently unforeseen or unanticipated alternatives, modifications, variations, or improvements therein may be subsequently made by those skilled in the art, each of which is also intended to be encompassed by the disclosed embodiments.

What is claimed is:

1. A method for the treatment of the pain associated with trigeminal neuralgia, comprising administering to a patient in need thereof a therapeutically effective amount of Trimeprazine or a pharmaceutically acceptable salt thereof, thereby treating the trigeminal neuralgia in the patient.

2. The method of claim 1, wherein the therapeutically effective amount of Trimeprazine or a pharmaceutically acceptable salt thereof is administered once a day.

3. The method of claim 1, wherein the therapeutically effective amount of Trimeprazine or a pharmaceutically acceptable salt thereof is administered twice a day.

4. The method of claim 1, wherein the therapeutically effective amount of Trimeprazine or a pharmaceutically acceptable salt thereof is administered via oral administration, intravenous administration (IV), intramuscular administration (IM), intrathecal administration, subcutaneous administration (SC), sublingual or buccal administration, rectal administration, vaginal administration, cutaneous administration, transdermal administration, nasal administration, or combinations thereof.

5. The method of claim 1, wherein the therapeutically effective amount of Trimeprazine or a pharmaceutically acceptable salt thereof is administered via oral administration.

6. The method of claim 1, wherein the therapeutically effective amount of Trimeprazine or a pharmaceutically acceptable salt thereof is administered via an oral thin film formulation.

7. The method of claim 1, wherein the therapeutically effective amount of Trimeprazine or a pharmaceutically acceptable salt thereof is about 0.5 mg to about 80 mg.

8. The method of claim 1, wherein the therapeutically effective amount of Trimeprazine or a pharmaceutically acceptable salt thereof is about 2.5 to about 5 mg.

9. The method of claim 1, wherein the therapeutically effective amount of Trimeprazine or a pharmaceutically acceptable salt thereof is about 0.5 to about 2.5 mg.

10. The method of claim 1, wherein the treatment of the pain associated with trigeminal neuralgia comprises a reduction in the frequency of pain, a reduction in the duration of pain, a reduction in the intensity of pain, or any combination thereof.

11. Then method of claim 10, wherein a reduction in the duration of pain comprises a reduction in the duration of a single episode of pain, a reduction in the duration of a series of episodes of pain, and any combination thereof.

12. The method of claim 11, wherein a series of episodes of pain comprises at least two separate episodes of pain.

13. The method of claim 1, wherein the pain associated with trigeminal neuralgia is stabbing pain, lancinating pain, shock-like pain, electric shock-like pain, constant aching, burning, stabbing pain of somewhat lower intensity than Type 1 TN, and any combination thereof.

14. The method of claim 11, wherein a reduction in the frequency of pain, a reduction in the duration of pain, a reduction in the intensity of pain, or any combination thereof is measured using a patient diary, a PGIC score, a MSQ score, a BNI Pain Intensity score, a Penn-FPS-R score, a Penn-FPS score, a EQ-5D-5L score, a WPAI score or any combination thereof.

15. The method of claim 1, wherein administering the therapeutically effective amount of Trimeprazine or a pharmaceutically acceptable salt thereof comprises administering an initial dose of between 2.5 to about 5.0 mg.

16. The method of claim 15, further comprising administering an additional therapeutically effective amount of Trimeprazine or a pharmaceutically acceptable salt thereof, about every 12 hours following the initial dose if treatment of pain associated with trigeminal neuralgia is not achieved.

17. The method of claim 16, wherein the additional, additional therapeutically effective amount of Trimeprazine or a pharmaceutically acceptable salt thereof is increased about every 30 minutes to 5 hours until treatment of the pain associated with trigeminal neuralgia is achieved.

18. The method of claim 17, wherein once treatment of pain associated with trigeminal neuralgia is achieved, the additional therapeutically effective amount of Trimeprazine or a pharmaceutically acceptable salt thereof is maintained as the minimum effective level used for the treatment of pain associated with trigeminal neuralgia.

19. The method of claim 18, wherein the minimum effective level of therapeutically effective amount of Trimeprazine or a pharmaceutically acceptable salt thereof is administered until treatment of pain treatment of pain associated with trigeminal neuralgia is no longer achieved, after which the minimum effective level of therapeutically effective amount of Trimeprazine or a pharmaceutically acceptable salt thereof may be further increased until treatment of pain treatment of pain associated with trigeminal neuralgia is once again achieved.

20. The method of claim 16, wherein at least about every 3 months, the therapeutically effective amount of Trimeprazine or a pharmaceutically acceptable salt thereof administered is decreased to the minimum effective level of therapeutically effective amount of Trimeprazine or a pharmaceutically acceptable salt thereof.

21. The method of claim 16, wherein at least about every 3 months, administration of the therapeutically effective amount of Trimeprazine or a pharmaceutically acceptable salt thereof administered is discontinued.
